(19) United States
(12) Patent Application Publication
(10) Pub. No.: US 2026/0357317 A1
(43) Pub. Date: Nov. 12, 2026

(54) GLUCURONOSYLTRANSFERASE, GENE ENCODING SAME AND METHOD FOR USING THE SAME

(71) Applicants: OSAKA UNIVERSITY, Suita-shi (JP); NATIONAL AGRICULTURE AND FOOD RESEARCH ORGANIZATION, Tsukuba-shi (JP)

(72) Inventors: Toshiya MURANAKA, Suita-shi (JP); Hikaru SEKI, Suita-shi (JP); Soo Yeon CHUNG, Suita-shi (JP); Masao ISHIMOTO, Tsukuba-shi (JP); Susumu HIRAGA, Tsukuba-shi (JP); Yukiko SATOU, Tsukuba-shi (JP)

(73) Assignees: OSAKA UNIVERSITY, Suita-shi (JP); NATIONAL AGRICULTURE AND FOOD RESEARCH ORGANIZATION, Tsukuba-shi (JP)

(21) Appl. No.: 19/690,339

(22) Filed: May 28, 2026

Related U.S. Application Data
(63) Continuation of application No. 17/769,627, filed on Oct. 16, 2020, now Pat. No. 12,692,527.

(30) Foreign Application Priority Data
Oct. 17, 2019 (JP) 2019-190060

(51) Int. Cl. C12P 19/56; C12N 1/16; C12N 9/10; C12N 15/81

(52) U.S. Cl. CPC C12P 19/56; C12N 1/16; C12N 9/1051; C12N 15/815; C12Y 204/01017; C12N 2800/102

(57) ABSTRACT

Glucuronosyltransferase 1 gene which catalyzes glucuronic acid transfer to the hydroxyl group at the 3-position in an oleanane-type triterpenoid is identified. Glucuronosyltransferase 1 gene having a desired activity, derived from a Fabaceae plant (soybean, Glycyrrhiza, and Lotus japonicus), and containing nucleotide sequences represented by SEQ ID Nos: 2, 4, and 6, respectively, is provided.

### CROSS-REFERENCE TO RELATED APPLICATIONS

The present application is a continuation application of U.S. application Ser. No. 17/769,627, filed on Sep. 1, 2022, which is a 35 U.S.C. § 371 national stage patent application of International patent application PCT/JP2020/039175, filed on Oct. 16, 2020, which is based on and claims the benefits of priority to Japanese Application No. 2019-190060, filed on Oct. 17, 2019. The entire contents of all of the above applications are incorporated herein by reference.

### REFERENCE TO A SEQUENCE LISTING

A Sequence Listing, submitted as an XML file and compliant with WIPO Standard ST.26, forms part of the present application. The Sequence Listing is identified as follows: File name “562698us.xml” created on May 27, 2026, with a size of 85,702 bytes. The Sequence Listing is incorporated by reference for all purposes.

### TECHNICAL FIELD

The present invention relates to an enzyme for transferring glucuronic acid to the hydroxyl group at the 3-position in an oleanane-type triterpenoid, a gene encoding the enzyme, and a method for producing glycyrrhizin.

### BACKGROUND ART

Glycyrrhiza (Glycyrrhiza uralensis) is a Fabaceae perennial herbaceous plant. Root and stolon of the plant are known as important Chinese herbal medicines (Kampo medicine), “licorice (Glycyrrhiza spp.)”, and widely used throughout the world. The main active ingredient contained in licorice is glycyrrhizin which is an oleanane-type triterpenoid saponin (Non-Patent Literature 1). Various studies have been conducted on glycyrrhizin for its usefulness in view of herbal medicine and pharmacology, thremmatological aspects, and the like.

In order to stably and continuously provide high-quality glycyrrhizin serving as a medicinal drug by a biological production system, for example, establishment of optimal production conditions by using a gene involved in a glycyrrhizin biosynthetic system or the expression level of the gene as a marker, selection of a high glycyrrhizin production strain, or breeding of the high glycyrrhizin production plant by introducing a synthase gene is required. Therefore, it is indispensable to identify group of genes involved in the glycyrrhizin biosynthetic pathway.

Glycyrrhizin is biologically synthesized from β-amyrin, which is commonly contained in plants and belongs to an oleanane-type triterpenoid, as a starting material, through two-step oxidation reactions and two-step glycosylation reactions. β-amyrin is known as a precursor substance, from which glycyrrhizin and soyasaponin I are biologically synthesized in a triterpenoid saponin biosynthetic pathway, in short, it serves as a branching point for biosynthesis of glycyrrhizin and soyasaponin I (FIG. 1).

As shown in FIG. 2, up to the present, as biosynthetic enzymes involved in the pathway from β-amyrin to glycyrrhizin, two types of oxidases have been known, namely, CYP88D6 (β-amyrin 11-oxidase; Patent Literature 1) and CYP72A154 (11-oxo-β-amyrin 30-oxidase; Patent Literature 2), which respectively catalyze each of the two-step oxidation reactions for biologically synthesizing glycyrrhetinic acid (which is an aglycone of glycyrrhizin) from β-amyrin. In addition, among the biosynthetic enzymes catalyzing two-step glycosylation reactions to biologically synthesize glycyrrhizin from the resulting glycyrrhetinic acid, a glycosyltransferase UGT73P12 (UDP-glycosyltransferase 73P12; Patent Literature 3), which catalyzes the second-step glycosylation reaction, has been known. Many skilled scientists in this field have repeatedly attempted to isolate glycosyltransferase, namely glucuronosyltransferase 1, which catalyzes the first-step of the glycosylation reaction of transferring glucuronic acid directly to glycyrrhetinic acid. Despite all their efforts, they could not have isolated the enzyme yet, which was a bottleneck in synthesizing glycyrrhizin from β-amyrin in a biological production system or in vitro synthetic system. Therefore, they have failed to stably and continuously provide a sufficient amount of glycyrrhizin.

### CITATION LIST

### Patent Literature

Patent Literature 1: Japanese Patent No. 5526323
Patent Literature 2: Japanese Patent No. 5771846
Patent Literature 3: Japanese Patent No. 6344774

### Non-Patent Literature

Non-Patent Literature 1: Gibson, M. R., 1978, Lloydia—the journal of Natural Products, 41(4): 348-354

### SUMMARY OF INVENTION

### Technical Problem

In order to solve the above-mentioned problem, the present invention is aimed at isolating a glucuronosyltransferase 1 gene, which catalyzes glucuronic acid transfer to the hydroxyl group at the 3-position of an oleanane-type triterpenoid including glycyrrhetinic acid. The present invention is also aimed at preparing and providing a biological expression system capable of biologically synthesizing a large amount of glycyrrhizin from j-amyrin in an individual or a cell using the gene expression system for glucuronosyltransferase 1.

### Solution to Problem

In the biosynthetic pathway from β-amyrin to glycyrrhizin, many skilled persons in the art have attempted to isolate the last remnant glucuronosyltransferase 1 from Glycyrrhiza. However, glucuronosyltransferase 1 could not have been isolated for many years. Therefore, the present inventors considered that there is some difficult factor in the isolation of glucuronosyltransferase 1 from Glycyrrhiza plants, and attempted to isolate the glucuronosyltransferase 1 from plants other than Glycyrrhiza plants. In Fabaceae plants to which Glycyrrhiza plants belong, there is generally a pathway for biologically synthesizing soyasaponin I from β-amyrin via soyasapogenol B as an intermediate product (FIG. 1). Soyasapogenol B, an oleanane-type triterpenoid, has a hydroxyl group at the 3-position, while the final product, soyasaponin I, has glucuronic acid bound at the 3-position. It is like a case where glycyrrhetinic acid, which is an intermediate product and is also an oleanane-type triterpenoid, has a hydroxyl group at the 3-position, whereas glycyrrhizin, which is a final product, has glucuronic acid bound at the 3-position. That is, glucuronosyltransferase 1 that functions in the biosynthetic pathway from β-amyrin to glycyrrhizin may also function in the biosynthetic pathway from β-amyrin to soyasaponin I. Based on the hypothesis, the present inventors attempted to isolate genes having glucuronic acid transfer activity 1 from soybeans (Glycine max) having a pathway for biologically synthesizing soyasaponin I from β-amyrin. As a result, the present inventors succeeded in isolating a cellulose synthase analogous gene whose specific function is unknown. The transglycosylation activity of this enzyme was verified using soyasapogenol B as a sugar acceptor substrate. As a result, it was found that glucuronic acid is transglycosylated to the hydroxyl group at the 3-position in soyasapogenol B. This enzyme activity was similar in a case where the sugar acceptor substrate is glycyrrhetinic acid. Thus, the present inventors shifted from Glycyrrhiza plants to soybean as a plant from which glucuronosyltransferase 1 is derived, and succeeded in identifying glucuronosyltransferase 1 which could not have been isolated before. The present invention is based on the research results, and provides the following.
(1) A polypeptide having an activity to transfer glucuronic acid to the hydroxy group at the 3-position in an oleanane-type triterpenoid, and comprising any one of amino acid sequences of the following (a) to (c), or a fragment thereof having the activity:
   (a) an amino acid sequence represented by any one of SEQ ID NOs: 1, 3, and 5,
   (b) an amino acid sequence derived from the amino acid sequence represented by any one of SEQ ID NOs: 1, 3, and 5 by deletion, replacement or addition of one or a plurality of amino acids, and
   (c) an amino acid sequence polypeptide having 80% or more identity with the amino acid sequence represented by any one of SEQ ID NOs: 1, 3, and 5.
(2) The polypeptide according to (1), wherein the oleanane-type triterpenoid is selected from the group consisting of β-amyrin, 11-oxo-β-amyrin, 30-hydroxy-11-oxo-β-amyrin, 30-hydroxy-β-amyrin, 24-hydroxy-β-amyrin, 11-deoxoglycyrrhetinic acid, glycyrrhetinic acid, oleanolic acid, medicagenic acid, soyasapogenol B, soyasapogenol A, hederagenin, camelliagenin, and saikogenin.
(3) The polypeptide according to (1) or (2), which is derived from a Fabaceae plant.
(4) A polynucleotide encoding the polypeptide according to any one of (1) to (3).
(5) The polynucleotide according to (4), comprising any one of nucleotide sequences of the following (a) to (d):
   (a) a nucleotide sequence represented by any one of SEQ ID NOs: 2, 4, and 6,
   (b) a nucleotide sequence derived from the nucleotide sequence represented by any one of SEQ ID NOs: 2, 4, and 6 by deletion, replacement or addition of one or a plurality of nucleotides,
   (c) a nucleotide sequence having 80% or more identity with the nucleotide sequence represented by any one of SEQ ID NOs: 2. 4, and 6, or
   (d) a nucleotide sequence capable of hybridizing with a complementary nucleotide sequence to the nucleotide sequence represented by any one of SEQ ID NOs: 2, 4, and 6 under high stringent conditions.
(6) A CSyGT (Cellulose synthase superfamily-derived glycosyltransferase 1) expression vector including the polynucleotide according to (4) or (5).
(7) A transformant comprising the polynucleotide according to (4) or (5) or the CSyGT expression vector according to (6), or a progeny thereof having the polynucleotide or the CSyGT expression vector.
(8) The transformant or a progeny thereof according to (7), wherein a host is a Fabaceae plant.
(9) The transformant or a progeny thereof according to (7), wherein a host is a yeast.
(10) The polypeptide according to any one of (1) to (3), prepared by adding a sugar chain derived from the yeast obtained from the transformant or a progeny thereof according to (9).
(11) The polypeptide according to (10), wherein the sugar chain derived from the yeast is high mannose-type sugar chain.
(12) A method for producing a polypeptide having an activity to transfer glucuronic acid to the hydroxy group at the 2-position of glucuronic acid in an oleanane-type triterpenoid, comprising the steps of culturing the transformant or a progeny thereof according to (7) or (8), and extracting the polypeptide according to any one of (1) to (3) from the culture.
(13) A gene recombinant for producing glycyrrhizin, capable of biologically synthesizing β-amyrin and comprising all expression vectors of the following (A) to (D):
(A) a CYP88D6 expression vector encoding a protein having an activity of oxidizing the 11-position in an oleanane-type triterpenoid and comprising a polypeptide containing any one of amino acid sequences of the following (a) to (c):
   (a) an amino acid sequence represented by SEQ ID NO: 7,
   (b) an amino acid sequence derived from the amino acid sequence represented by SEQ ID NO: 7 by deletion, replacement or addition of one or a plurality of amino acids, and
   (c) an amino acid sequence having 80% or more identity with the amino acid sequence represented by SEQ ID NO: 7,
(B) a CYP72A154 expression vector encoding a protein having an activity of oxidizing the 30-position in an oleanane-type triterpenoid and comprising a polypeptide containing any one of amino acid sequences of the following (d) to (f):
   (d) an amino acid sequence represented by any one of SEQ ID NOs: 9, 11 and 13,
   (e) an amino acid sequence derived from the amino acid sequence represented by any one of SEQ ID NOs: 9, 11, and 13 by deletion, replacement or addition of one or a plurality of amino acids, or
   (f) an amino acid sequence having 80% or more identity with the amino acid sequence represented by any one of SEQ ID NOs: 9, 11, and 13,
(C) an UGT73P12 expression vector encoding a protein having an activity to transfer glucuronic acid to the hydroxy group at the 2-position of glucuronic acid in an oleanane-type triterpenoid monoglucuronide and comprising a polypeptide containing any one of amino acid sequences of the following (g) to (i):
   (g) an amino acid sequence represented by SEQ ID NO: 15,
   (h) an amino acid sequence derived from the amino acid sequence represented by SEQ ID NO: 15 by deletion, replacement or addition of one or a plurality of amino acids, or
   (i) an amino acid sequence having 80% or more identity with the amino acid sequence represented by SEQ ID NO: 15, and
(D) a CSyGT expression vector according to (6).
(14) The gene recombinant according to (13), wherein a host is a Fabaceae plant.
(15) A method for producing glycyrrhizin from β-amyrin, comprising a step of culturing the gene recombinant according to (13) or (14).

The specification incorporates the contents disclosed in the specification of Japanese Patent Application No. 2019-190060 based on which the priority of the present application is claimed.

### Advantageous Effects of Invention

According to the present invention, there is provided a polypeptide having an activity to transfer glucuronic acid to the hydroxy group at the 3-position in an oleanane-type triterpenoid, a polynucleotide encoding the polypeptide, or use thereof.

### BRIEF DESCRIPTION OF DRAWINGS

FIG. 1 shows biosynthetic pathways of glycyrrhizin and soyasaponin I in a triterpenoid saponin biosynthetic system.

FIG. 2 shows oleanane-type triterpenoids generated as intermediate products in biosynthetic pathways from β-amyrin to glycyrrhizin, known enzymes (CYP88D6, CYP72A154, and UGT73P12) involved in conversion reactions, and the steps catalyzed by glucuronosyltransferase 1 CSyGT newly identified in the specification.

FIG. 3 is a conceptual diagram showing triterpenoid producing yeast strains used for functional analyses of soybean Glyma.06G324300, and Glycyrrhiza- and Lotus japonicus-derived Glyma.06G324300 homologous proteins in examples. The yeast strain in (a) can intracellularly produce glycyrrhetinic acid from 2,3-oxidosqualene endogenously in yeast by co-expression of 3-amyrin synthase gene (Lotus japonicus-derived), CYP88D6 (Glycyrrhiza-derived), and CYP72A63 (M. truncatula-derived). The yeast strain in (b) can intracellularly produce soyasapogenol B from 2,3-oxidosqualene endogenously in yeast by co-expression of β-amyrin synthase gene (Lotus japonicus-derived), CYP93E3 (Glycyrrhiza-derived), and CYP72A566 (Glycyrrhiza-derived).

FIG. 4 shows metabolite analysis results of glycyrrhetinic acid producing yeast, into which Glyma.06G324300 and homologous gene were introduced. Sample A in (a) shows detection results of glycyrrhetinic acid monoglucuronide generated by expressing soybean-derived Glyma.06G324300 in glycyrrhetinic acid-producing yeast; Sample B in (b) shows detection results of glycyrrhetinic acid monoglucuronide generated by expressing Glycyrrhiza-derived Glyur003152s00037491 in glycyrrhetinic acid-producing yeast; Sample C in (c) shows detection results of glycyrrhetinic acid monoglucuronide generated by expressing Lotus japonicus-derived Lj3g3v1981230 in glycyrrhetinic acid-producing yeast. The peak pointed by a solid arrow shows glycyrrhetinic acid monoglucuronide. Sample D in (d) shows detection results in glycyrrhetinic acid-producing yeast, into which an empty vector was introduced as a negative control.

FIG. 5 shows metabolite analysis results of soyasapogenol B producing yeast, into which Glyma.06G324300 and homologous gene were introduced. Sample E in (a) shows detection results of soyasapogenol B monoglucuronide generated by expressing soybean-derived Glyma.06G324300 in soyasapogenol B-producing yeast; Sample F in (b) shows detection results of soyasapogenol B monoglucuronide generated by expressing Glycyrrhiza-derived Glyur003152s00037491 in soyasapogenol B-producing yeast; Sample G in (c) shows detection results of soyasapogenol B monoglucuronide generated by expressing Lotus japonicus-derived Lj3g3v1981230 in soyasapogenol B-producing yeast. The peak pointed by a solid arrow shows soyasapogenol B monoglucuronide. Sample H in (d) shows detection results in soyasapogenol B-producing yeast in which an empty vector was introduced as a negative control.

FIG. 6 shows an overview of the substrate feeding experiment. Arabidopsis thaliana UDP-glucose dehydrogenase (AtUGD), Glyma.06G324300, and homologous genes thereof were co-expressed in the yeast to intracellularly supply UDP-glucuronic acid which is a sugar donor substrate. (a) shows a conversion reaction to monoglucuronide when glycyrrhetinic acid was added to the yeast culture, and (b) shows a conversion reaction to monoglucuronide when soyasapogenol B was added to the yeast culture.

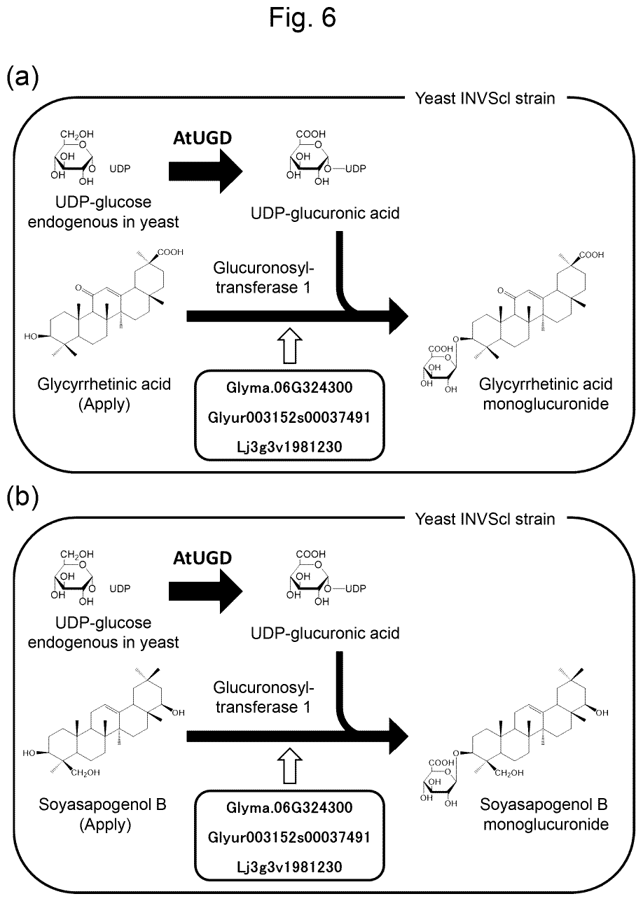

FIG. 7 shows metabolite analysis results in soybean-derived Glyma.06G324300-introduced yeast, to which glycyrrhetinic acid was fed. (a) is a conceptual diagram showing envisaged conversion reactions to glycyrrhetinic acid monoglucuronide when Glyma.06G324300 is used. (b) shows detection results of glycyrrhetinic acid before conversion. The peak pointed by an open arrow shows glycyrrhetinic acid. (c) shows detection results of glycyrrhetinic acid monoglucuronide generated by Glyma.06G324300. The peak pointed by a solid arrow shows glycyrrhetinic acid monoglucuronide.

FIG. 8 shows metabolite analysis results in Glycyrrhiza-derived Glyur003152s00037491-introduced yeast, to which glycyrrhetinic acid was fed. (a) is a conceptual diagram showing envisaged conversion reactions to glycyrrhetinic acid monoglucuronide when Glyur003152s00037491 is used. (b) shows detection results of glycyrrhetinic acid before conversion. The peak pointed by an open arrow shows glycyrrhetinic acid. (c) shows detection results of glycyrrhetinic acid monoglucuronide generated by Glyur003152s00037491. The peak pointed by a solid arrow shows glycyrrhetinic acid monoglucuronide.

FIG. 9 shows metabolite analysis results in Lotus japonicus-derived Lj3g3v1981230-introduced yeast, to which glycyrrhetinic acid was fed. (a) is a conceptual diagram showing envisaged conversion reactions to glycyrrhetinic acid monoglucuronide when Lj3g3v1981230 is used. (b) shows detection results of glycyrrhetinic acid before conversion. The peak pointed by an open arrow shows glycyrrhetinic acid. (c) shows detection results of glycyrrhetinic acid monoglucuronide generated by Lj3g3v1981230. The peak pointed by a solid arrow shows glycyrrhetinic acid monoglucuronide.

FIG. 10 shows metabolite analysis results in the empty vector-introduced yeast, to which glycyrrhetinic acid was fed, as the negative controls of FIGS. 7 to 9. (a) is a conceptual diagram showing envisaged conversion reactions to glycyrrhetinic acid monoglucuronide when the empty vector is used. It is expected that no glycyrrhetinic acid monoglucuronide is produced due to lack of enzyme activity. (b) shows detection results of glycyrrhetinic acid before reaction. The peak pointed by an open arrow shows glycyrrhetinic acid. (c) shows detection results of glycyrrhetinic acid monoglucuronide after reaction. The peak pointed by a solid arrow shows glycyrrhetinic acid monoglucuronide. A dash line arrow shows a position at which the peak was generated when glycyrrhetinic acid monoglucuronide was produced.

FIG. 11 shows metabolite analysis results in Glyma.06G324300 and its homologous gene-introduced yeast, to which soyasapogenol B was fed. (a) is a conceptual diagram showing envisaged conversion reactions from soyasapogenol B to soyasapogenol B monoglucuronide. (b) shows detection results of soyasapogenol B monoglucuronide generated by soybean-derived Glyma.06G324300. The peak pointed by a solid arrow shows soyasapogenol B monoglucuronide. (c) shows detection results of soyasapogenol B monoglucuronide generated by Glycyrrhiza-derived Glyur003152s00037491. The peak pointed by a solid arrow shows soyasapogenol B monoglucuronide. (d) shows detection results of soyasapogenol B monoglucuronide generated by Lotus japonicus-derived Lj3g3v1981230. The peak pointed by a solid arrow shows soyasapogenol B monoglucuronide. (e) shows negative controls of (b) to (d) described above.

FIG. 12-1 shows LC-PDA/MS/MS analysis results of Lotus japonicus Glyma.06G324300 homologous gene loss-of-function mutant extract. (a) shows a base peak ion chromatogram of a plant extract derived from wild-type (Gifu), and (b) and (c) show base peak ion chromatograms of plant extracts derived from transposon insertion homozygous mutants (30006020 and 30115796, respectively).

FIG. 12-2 shows LC-PDA/MS/MS analysis results of Lotus japonicus-Glyma.06G324300 homologous gene loss-of-function mutant extract. (a) shows a total ion chromatogram of a plant extract derived from wild-type (Gifu), and (b) and (c) show total ion chromatograms of plant extracts derived from transposon insertion homozygous mutants (30006020 and 30115796, respectively).

FIG. 13 shows LC-PDA/MS/MS analysis results of Glyma.06G324300 homologous gene-introduced loss-of-function mutant hairy root. This figure shows total ion chromatograms of hairy root extracts derived from: (a) a strain of wild-type (Gifu) into which an empty vector (pG35N_empty) was introduced, and (b) to (e) transformants of Glyma.06G324300 homologous gene loss-of-function homozygous mutant (30115796) strains into which pG35N_empty, expression vectors pG35N-GmCSL, pG35N-GuCSL, and pG35N-LjCSL were introduced, respectively.

FIG. 14 shows metabolite analysis results in Astragalus Glyma.06G324300 orthologue gene- and soybean Glyma.06G324300 paralogue gene-introduced glycyrrhetinic acid-producing yeast. Sample Q in (a) shows detection results of glycyrrhetinic acid monoglucuronide generated by expressing an AsCSyGT gene, which is an Astragalus-derived Glyma.06G324300 orthologue gene, in glycyrrhetinic acid-producing yeast; Sample R in (b) shows detection results of glycyrrhetinic acid monoglucuronide generated by expressing a Glyma04g255400 gene, which is a soybean-derived Glyma.06G324300 paralogue gene, in glycyrrhetinic acid-producing yeast; Sample S in (c) shows detection results of glycyrrhetinic acid monoglucuronide generated by expressing a Glyma.11g151800 gene, which is a soybean-derived Glyma.06G324300 paralogue gene, in glycyrrhetinic acid-producing yeast. The peak pointed by a solid arrow shows glycyrrhetinic acid monoglucuronide.

FIG. 15 shows metabolite analysis results in soyasapogenol B-producing yeast, into which Astragalus Glyma.06G324300 orthologue gene and soybean Glyma.06G324300 paralogue gene were introduced. Sample T in (a) shows detection results of soyasapogenol B monoglucuronide generated by expressing an Astragalus-derived AsCSyGT gene in soyasapogenol B-producing yeast; Sample U in (b) shows detection results of soyasapogenol B monoglucuronide generated by expressing a soybean-derived Glyma04g255400 gene in soyasapogenol B-producing yeast; Sample V in (c) shows detection results of soyasapogenol B monoglucuronide generated by expressing a soybean-derived Glyma.11g151800 gene in soyasapogenol B-producing yeast. The peak pointed by a solid arrow shows soyasapogenol B monoglucuronide.

FIG. 16 shows metabolite analysis results in soybean-derived Glyma.11g151800 gene-introduced yeast, to which ursolic acid was fed. (a) is a conceptual diagram showing envisaged conversion reactions to ursolic acid monoglucuronide when Glyma.11g151800 feeding assay extract (sample W) was used. (b) shows detection results of ursolic acid monoglucuronide generated by Glyma.11g151800 when sample W was added. The peak pointed by a solid arrow shows ursolic acid monoglucuronide. (c) shows detection results of ursolic acid monoglucuronide when sample X as a negative control was added.

FIG. 17 shows metabolite analysis results in soybean-derived Glyma.11g151800 gene-introduced yeast, to which betulinic acid was fed. (a) is a conceptual diagram showing envisaged conversion reactions to betulinic acid monoglucuronide when Glyma.11g151800 feeding assay extract (sample Y) was used. (b) shows detection results of betulinic acid monoglucuronide generated by Glyma.11g151800 when sample Y was added. The peak pointed by a solid arrow shows betulinic acid monoglucuronide. (c) shows detection results of betulinic acid monoglucuronide when sample Z as a negative control was added.

### DESCRIPTION OF EMBODIMENTS

### 1. Glucuronosyltransferase 1 (CSyGT)

### 1-1. Summary

A first aspect of the present invention relates to glucuronosyltransferase 1 and a fragment thereof having glucuronic acid transfer activity 1, and nucleic acid encoding them. The glucuronosyltransferase 1 of the present invention has an activity to catalyze a glycosylation reaction of glucuronic acid to the hydroxyl group at the 3-position of glycyrrhetinic acid which is generated from β-amyrin via two-step oxidation reaction, in a synthetic pathway peculiar to the genus Glycyrrhiza that biologically synthesizes glycyrrhizin from β-amyrin which can be biologically synthesized by many plants. The glucuronosyltransferase 1 of the present invention or the like enables not only sugar transfer of glucuronic acid to the hydroxyl group at the 3-position of oleanane-type triterpenoids including glycyrrhetinic acid, but also creation of a biological production system from B-amyrin to glycyrrhizin using a plant other than a Glycyrrhiza plant as a host, by combining with known enzyme involving in a biosynthetic pathway from β-amyrin to glycyrrhizin. Thereby, high quality glycyrrhizin can be stably and continuously provided.

### 1-2. Definition of Terms

The terms frequently used herein are defined as follows:

In the specification, the “glucuronosyltransferase 1 (CSyGT)” (often referred to as “CSyGT” in the specification) refers to an enzyme catalyzing a sugar transfer reaction to an oleanane-type triterpenoid having the hydroxyl group at the 3-position carbon, the sugar transfer reaction including transferring a single glucuronic acid, a kind of sugar, to the hydroxyl group of the oleanane-type triterpenoid. The term “first” as used herein means to have the first transglycosylation activity in a two-step sugar transfer reaction at the hydroxyl group at the 3-position of the oleanane-type triterpenoid. The specific configuration of glucuronosyltransferase 1 will be described later.

In the specification, “fragment thereof having glucuronic acid transfer activity 1” refers to an active fragment of the glucuronosyltransferase 1.

In the specification, “glucuronic acid transfer activity 1” refers to an activity of the CSyGT to catalyze a sugar transfer reaction, that is, an activity to transfer glucuronic acid to the hydroxyl group at the 3-position in an oleanane-type triterpenoid. Owing to the activity, an oleanane-type triterpenoid monoglucuronide is produced from an oleanane-type triterpenoid. Note that, in the specification, CSyGT and a fragment thereof having glucuronic acid transfer activity 1 are often collectively referred to as “polypeptide having glucuronic acid transfer activity 1” or “glucuronosyltransferase 1 or the like (CSyGT or the like)”. Note that, in the specification, glucuronosyltransferase 1 and the like may be a glycoprotein prepared by adding different sugar chains. For example, glucuronosyltransferase 1 and the like prepared by adding a plant-derived sugar chain, and glucuronosyltransferase 1 and the like prepared by adding a yeast-derived sugar chain are both included in glucuronosyltransferase 1 and the like in the specification.

The “oleanane-type triterpenoid” refers to C30 isoprenoid having a pentacyclic oleanane skeleton and comprised of 6 isoprene units, and corresponds to aglycone of glycyrrhizin which is a final objective product in the invention. The oleanane-type triterpenoid described in the specification refers to an oleanane-type triterpenoid having the hydroxyl group (OH group) at the 3-position, unless otherwise indicated. Specific examples of the oleanane-type triterpenoid are not limited, but include β-amyrin, 11-oxo-β-amyrin, 30-hydroxy-11-oxo-β-amyrin, 30-hydroxy-β-amyrin, 24-hydroxy-β-amyrin, 11-deoxoglycyrrhetinic acid, glycyrrhetinic acid, oleanolic acid, medicagenic acid, soyasapogenol B, soyasapogenol A, hederagenin, camelliagenin, and saikogenin. All of them can be substrates for glucuronosyltransferase 1 of the present invention. Owing to glucuronic acid transfer activity, β-amyrin-3-O-monoglucuronide, 11-oxo-β-amyrin-3-O-monoglucuronide, 30-hydroxy-11-oxo-β-amyrin-3-O-monoglucuronide, 30-hydroxy-β-amyrin-3-O-monoglucuronide, 24-hydroxy-β-amyrin-3-O-monoglucuronide, 11-deoxoglycyrrhetinic acid-3-O-monoglucuronide, glycyrrhetinic acid-3-O-monoglucuronide, oleanolic acid-3-O-monoglucuronide, medicagenic acid-3-O-monoglucuronide, soyasapogenol B-3-O-monoglucuronide, soyasapogenol A-3-O-monoglucuronide, hederagenin-3-O-monoglucuronide, camelliagenin-3-O-monoglucuronide, and saikogenin-3-O-monoglucuronide are biologically synthesized from the substrate.

In the specification, Fabaceae plants are not limited to Glycyrrhiza plants, but include all plant species belonging to the Fabaceae family according to plant taxonomy. Examples thereof include an Arachis plant, a Cicer plant, an Aspalathus plant, a Dalbergia plant, a Pterocarpus plant, a Desmodium plant, a Lespedeza plant, an Uraria plant, a Galegeae plant, an Astragalus plant, a Glycyrrhiza plant, an Oxytropis plant, an Augyrocytisus plant, a Cytisus plant, a Genista plant, a Spartium plant, a Hedysarum plant, a Cyamopsis plant, an Indigofera plant, a Lotus japonicus (Lotus) plant, a Lupinus plant, a Wisteria plant, a Cajanus plant, a Canavalia plant, an Erythrina plant, a soybean (Glycine) plant, a Hardenbergia plant, a Lablab plant, a Mucuna plant, a Phaseolus plant, a Psophocarpus plant, a Pueraria plant, a Vigna plant, a Robinia plant, a Castanospermum plant, a Maackia plant, an Ormosia plant, a Sophora plant, a Styphnolobium plant, a Medicago plant, a Trigonella plant, a Trifolium plant, a Lathyrus plant, a Lens plant, a Pisum plant and a Vicia plant. Glycyrrhiza plants to which G. uralensis belongs, and their related species, Medicago plants, have a biosynthetic pathway for glycyrrhetinic acid which is a substrate of CSyGT in biologically synthesizing glycyrrhizin. Therefore, they are suitable for Fabaceae plants of the present invention. Specific examples of the Glycyrrhiza plant include G. glabra, G. inflata, G. aspera, G. eurycarpa, G. pallidiflora, G. yunnanensis, G. lepidota, G. echinata, and G. acanthocarpa. Specific examples of the Medicago plant include M truncatula.

### 1-3. Configuration

The glucuronosyltransferase 1 of the present invention (CSyGT) is a polypeptide consisting of amino acid sequences represented by any one of SEQ ID NOs: 1, 3, and 5. These polypeptides respectively corresponds to soybean (Glycine max) derived wild-type CSyGT(GmCSyGT), Glycyrrhiza (G. uralensis) derived wild-type CSyGT (GuCSyGT), and Lotus japonicus-(Lotus japonicus) derived wild-type CSyGT(LjCSyGT). Glycyrrhiza-derived GuCSyGT has 81% amino acid identity with soybean-derived GmCSyGT. Lotus japonicus-derived LjCSyGT has 82% amino acid identity with soybean-derived GmCSyGT.

CSyGT may have orthologs in many other plant species other than the above described plant species, especially in the Fabaceae plant species. The CSyGT of the present invention includes, in addition to such heterologous wild-type CSyGT orthologs, homologous wild-type CSyGT paralogs and mutant CSyGT that have glucuronic acid transfer activity 1. Examples of such heterologous wild-type CSyGT orthologs and mutant CSyGT include amino acid sequence derived from the amino acid sequence represented by any one of SEQ ID NOs: 1, 3, and 5 by deletion, replacement or addition of one or a plurality of amino acids, or polypeptides having 80% or more, 82% or more, 85% or more, 87% or more, 90% or more, 91% or more, 92% or more, 93% or more, 94% or more, 95% or more, 96% or more, 97% or more, 98% or more, or 99% or more and less than 100% amino acid identity with the amino acid sequence represented by any one of SEQ ID NOs: 1, 3, and 5. In fact, as opposed to soybean GmCSyGT, GuCSyGT and LjCSyGT are Glycyrrhiza and Lotus japonicus CSyGT orthologs, respectively, and have 80% or more amino acid identity as described above. Specific examples of mutant CSyGT having glucuronic acid transfer activity 1 includes without limitation splicing variants, mutants based on SNPs.

In the specification, the term “plurality” refers to, for example, 2 to 20, 2 to 15, 2 to 10, 2 to 7, 2 to 5, 2 to 4 or 2 to 3. “Amino acid identity” refers to the ratio (%) of the number of matched amino acid residues to the total number of amino acid residues in the amino acid sequences of the two polypeptides to be compared, when aligned by inserting gaps in one or both as necessary to maximize the number of matched amino acid residues. Alignment of the two amino acid sequences for calculating amino acid identity can be done using known programs such as Blast, FASTA, ClustalW, etc.

In the specification, “replacement (of amino acid)” refers to replacement within a group of conservative amino acids having similar properties such as charge, side chains, polarity, and aromaticity, among 20 amino acids constituting naturally occurring protein. Examples include replacement within a group of uncharged polar amino acids having slightly polar sidechains (Gly, Asn, Gln, Ser, Thr, Cys, Tyr), replacement within a group of branched chain amino acids (Leu, Val, Ile), replacement within a group of neutral amino acids (Gly, Ile, Val, Leu, Ala, Met, Pro), replacement within a group of neutral amino acids having hydrophilic side chains (Asn, Gln, Thr, Ser, Tyr, Cys), replacement within a group of acidic amino acids (Asp, Glu), replacement within a group of basic amino acids (Arg, Lys, His), and replacement within a group of aromatic amino acid(Phe, Tyr, Trp). Amino acid replacements within these groups are preferred because they are known to be less likely to cause changes in the properties of polypeptides.

In the present aspect, the “active fragment thereof” refers to a polypeptide fragment containing a region of glucuronosyltransferase 1 and having glucuronic acid transfer activity 1. An example is a polypeptide fragment containing a substrate-binding site of glucuronosyltransferase 1. An example of the substrate of the glucuronosyltransferase 1 of the present invention includes the above-described oleanane-type triterpenoid. Glycyrrhetinic acid is preferred. The length of the amino acids of the polypeptide constituting the active fragment is not particularly limited. The region may consist of continuous amino acids of, for example, at least 10, 15, 20, 25, 30, 50, 100 or 150 in (a) to (c) polypeptides.

Note that, in the specification, CSyGT and an active fragment thereof are often collectively referred to as “CSyGT and the like (glucuronosyltransferase 1 and the like)”.

According to the CSyGT or the like of the present invention, an oleanane-type triterpenoid monoglucuronide can be obtained by transglycosylating glucuronic acid to the hydroxyl group at the 3-position, using an oleanane-type triterpenoid as a sugar acceptor substrate by glucuronic acid transfer activity 1.

In the biosynthetic system of glycyrrhizin in Glycyrrhiza, the pathway for biosynthesis from β-amyrin, which is an oleanane-type triterpenoid and also said to be an original substance, to glycyrrhetinic acid has already been known, and it is also possible to be artificially biosynthesized. Further, the pathway for biologically synthesizing glycyrrhizin, in which glucuronic acid is further transglycosylated to glycyrrhetinic acid monoglucuronide prepared by transglycosylating one molecule of glucuronic acid to glycyrrhetinic acid, has also been known. That is, in the pathway for biologically synthesizing glycyrrhizin from β-amyrin, only the pathway of biologically synthesizing glycyrrhetinic acid monoglucuronide by transglycosylating glucuronic acid to glycyrrhetinic acid has been unknown. However, the present invention has revealed all of the biosynthetic pathways of glycyrrhizin in Glycyrrhiza, which enables in vitro synthesis from β-amyrin to glycyrrhizin. Further, since β-amyrin can be biologically synthesized from a lot of plant species other than Glycyrrhiza, in vivo synthesis system using ordinary ones among such plant species as a host can also be utilized. Furthermore, an organism can be used together with a gene biologically synthesizing β-amyrin so as to enable in vivo synthesis system using the organism as a host, even if the organism is other than a plant, as long as the organism does not contain β-amyrin but can biologically synthesize a precursor of β-amyrin.

### 2. Glucuronosyltransferase 1 Gene (CSyGT Gene) and Active Fragment Thereof

### 2-1. Summary

A second aspect of the present invention relates to a polynucleotide encoding the polypeptide (CSyGT and the like) described in the first aspect, that is, a glucuronosyltransferase 1 gene and an active fragment thereof. The polynucleotide of the present invention enables the construction of the recombinant vector of the third aspect described below.

### 2-2. Configuration

The “glucuronosyltransferase 1 gene” (often referred to herein as “CSyGT gene”) refers to a polynucleotide encoding CSyGT described in the first aspect. The nucleotide sequence of the polynucleotide is not particularly limited as long as the polynucleotide encodes CSyGT. The polynucleotide is preferably a polynucleotide encoding a wild-type CSyGT containing amino acid sequence represented by SEQ ID NOs: 1, 3 or 5. Examples include a polynucleotide encoding soybean-derived wild-type GmCSyGT consisting of the amino acid sequence represented by SEQ ID NO: 1, specifically for example, a polynucleotide consisting of the nucleotide sequence represented by SEQ ID NO: 2, that is, soybean-derived wild-type GmCSyGT gene. Further, examples include a polynucleotide encoding Glycyrrhiza-derived wild-type GuCSyGT consisting of the amino acid sequence represented by SEQ ID NO: 3, specifically for example, a polynucleotide consisting of the nucleotide sequence represented by SEQ ID NO: 4, that is, Glycyrrhiza-derived wild-type GuCSyGT gene. In addition, examples include a polynucleotide encoding Lotus japonicus-derived wild-type LjCSyGT consisting of the amino acid sequence represented by SEQ ID NO: 5, specifically for example, a polynucleotide consisting of the nucleotide sequence represented by SEQ ID NO: 6, that is, Lotus japonicus-derived wild-type LjCSyGT gene.

In addition, heterologous ortholog of the wild-type CSyGT gene, and mutant CSyGT genes maintaining the enzyme activity are also included. Examples of such CSyGT genes include polynucleotides containing nucleotide sequences derived from the wild-type CSyGT gene by deletion, replacement or addition of one or a plurality of nucleotides. Specifically, included are, for example, polynucleotides containing nucleotide sequences derived from any nucleotide of soybean-derived wild-type GmCSyGT gene (for example, polynucleotide consisting of the nucleotide sequence represented by SEQ ID NO: 2), Glycyrrhiza-derived wild-type GuCSyGT gene (for example, polynucleotide consisting of the nucleotide sequence represented by SEQ ID NO: 4), and Lotus japonicus-derived wild-type LjCSyGT gene (for example, polynucleotide consisting of the nucleotide sequence represented by SEQ ID NO: 6) by deletion, replacement, or addition of one or a plurality of nucleotides.

Further, examples include polynucleotides containing nucleotide sequences having 80% or more, 85% or more, 87% or more, 90% or more, 95% or more, or 99% or more and less than 100% amino acid identity of nucleotide identity with the wild-type CSyGT gene. Specifically, included are, for example, polynucleotides containing nucleotide sequence having 80% or more, 85% or more, 87% or more, 90% or more, 95% or more, or 99% or more and less than 100% nucleotide identity with nucleotide sequence of any one of soybean-derived wild-type GmCSyGT gene (for example, polynucleotide consisting of the nucleotide sequence represented by SEQ ID NO: 2), Glycyrrhiza-derived wild-type GuCSyGT gene (for example, a polynucleotide consisting of the nucleotide sequence represented by SEQ ID NO: 4), and Lotus japonicus-derived wild-type LjCSyGT gene (for example, polynucleotide consisting of the nucleotide sequence represented by SEQ ID NO: 6).

Also included is a polynucleotide containing a nucleotide sequence which hybridizes with a nucleotide fragment consisting of nucleotide sequence complementary to the partial nucleotide sequence of the wild-type CSyGT gene under high stringent conditions, and having the enzyme activity.

In the specification, “stringent condition” means a condition under which a non-specific hybrid is less likely to be formed. In the specification, “high stringent condition” refers to a condition under which a non-specific hybrid is still less likely to be formed, or is not formed. Generally, in the reaction condition, the lower the salt concentration and the higher the temperature, the higher the stringent conditions. For example, it corresponds to, for example, a condition of washing with 0.1×SSC and 0.1% SDS at 50 °C to 70 °C, 55 °C to 68 °C, or 65 °C to 68 °C in washing after hybridization. In addition, stringency of hybridization can be increased by appropriately combining other conditions such as probe concentration, probe nucleotide length, and hybridization time.

In the present aspect, “active fragment thereof” refers to a fragment of CSyGT gene, in which a polypeptide encoded by the fragment has CSyGT activity. Substantially, a polynucleotide encoding the active fragment of CSyGT described in the first aspect corresponds thereto. Therefore, the length of the nucleotide sequence of the polynucleotide constituting the active fragment, that is, the nucleotide number may be three time the amino acid sequence in the active fragment of CSyGT described in the first aspect.

According to the polynucleotide of the present invention or an active fragment thereof, a recombinant vector capable of expressing CSyGT and an active fragment thereof in a host cell can be constructed.

Note that, in the specification, CSyGT gene and an active fragment thereof are often collectively referred to as “CSyGT gene or the like (glucuronosyltransferase 1 gene or the like)”.

The CSyGT gene and the like of the present aspect can be isolated from a suitable plant, for example, a Fabaceae plant by a known method. For example, based on the nucleotide sequence of the soybean-derived wild-type GmCSyGT gene represented by SEQ ID NO: 2, a primer pair having an appropriate nucleotide sequence length is designed. A specific example includes the primer pair represented by SEQ ID NOs: 17 and 18. The GmCSyGT gene can be obtained by using the pair and performing a nucleic acid amplification reaction such as PCR using a nucleic acid derived from soybean DNA library or genomic DNA library as a template. The polynucleotide of the present invention can be also obtained from the library or the like by hybridization using a nucleic acid fragment consisting of a part of the nucleotide sequence represented by SEQ ID NO: 2 as a probe. As to these methods, the methods described in Green & Sambrook, Molecular Cloning, 2012, Fourth Ed., Cold Spring Harbor Laboratory Press may be referred to.

### 3. Recombinant Vector

### 3-1. Summary

A third aspect of the present invention relates to a recombinant vector. The recombinant vector of the present invention contains the polynucleotide described in the second aspect, and is capable of cloning CSyGT gene and the like or expressing CSyGT and the like in a host cell. In the present aspect, in particular, CSyGT expression vectors expressing CSyGT and the like are preferably applicable.

### 3-2. Configuration

The recombinant vector of the present invention can be constructed by introducing the polynucleotide described in the second aspect into an appropriate recombinant vector. The type of vector is not particularly limited and may appropriately be selected depending upon the purpose such as cloning (transformation) or gene expression, or depending upon the host to be introduced. A plant transformation vector or (gene) expression vector is particularly preferable.

In the present invention, “(gene) expression vector” is a gene expression system capable of transporting a polynucleotide encoding an included polypeptide into a target plant cell and expressing the polypeptide. An example includes an expression vector utilizing a plasmid or a virus. In the present invention, CSyGT expression vectors expressing CSyGT and the like integrating CSyGT gene and the like corresponds thereto.

In a case of expression vectors utilizing plasmids (hereinafter often referred to as “plasmid expression vector”), as a plasmid, for example but not limited to, pPZP-based, pSMA-based, pUC-based, pBR-based, pBluescript-based (Agilent Technologies), pTriEX™-based (TAKARA BIO INC.), or pBI-based, pRI-based or pGW-based binary vector or the like may be utilized.

In a case of expression vectors utilizing viruses (hereinafter often referred to as “virus expression vector”), as a virus, cauliflower mosaic virus (CaMV), bean golden mosaic virus (BGMV), tobacco mosaic virus (TMV) or the like can be used.

The recombinant vector includes expression regulatory regions of promoter and terminator. In addition, the recombinant vector can also include enhancers, poly-A addition signals, 5′-UTR (untranslated region) sequences, labelled or selective marker genes, multi-cloning sites, replication origins, and the like. Types of those mentioned above are not particularly limited so long as they can exert their function in a host cell. Those known in the art can be appropriately selected depending on the host into which they are introduced. The case where a plant cell or a plant is used as a host is preferable.

As a promoter, various promoters such as overexpressed promoters, constitutive promoters, site-specific promoters, time-specific promoters, and/or inducible promoters can be used. Specific examples of an overexpressed and constitutive promoter, which is operatable in plant cells include a cauliflower mosaic virus (CaMV)-derived 35S promoter, a promoter of a Ti plasmid-derived nopaline synthase gene (Pnos), a corn-derived ubiquitin promoter, a rice plant-derived actin promoter, and a tobacco-derived PR protein promoter and the like. Small subunits (Rubisco ssu) promoters of ribulose diphosphate carboxylase of various plant species, or histone promoters can also be used. As a workable promoter in bacterial cells, e.g., a promotor of a maltogenic amylase gene of Bacillus stearothermophilus, u-amylase gene of Bacillus lichenformis, a BAN amylase gene of Bacillus amyloliquefaciens, an alkaliprotease gene of Bacillus subtillis or a xylosidase gene of Bacillus pumilus; a PR or PL promoter of phage lambda; or lac, trp or tac promoter of E. coli and the like is mentioned. As a workable promoter in yeast host cells, e.g., a promoter derived from a yeast glycolytic gene, an alcohol dehydrogenase gene promoter, a TPI1 promoter or an ADH2-4c promoter and the like is mentioned. As a workable promoter in fungus, an ADH3 promoter or a tpiA promoter and the like is mentioned. As a workable promoter in animal cells, an SV40 early promoter, an SV40 late promotor or a CMV promoter and the like is mentioned. As a workable promoter in insect cells, a polyhedrin promoter, a P10 promoter, a basic protein promoter of a baculovirus, i.e., Autographa californica polyhedrosis, a baculovirus immediate early gene 1 promoter or a baculovirus 39K delayed early gene promoter and the like is mentioned.

As a terminator, for example, a terminator of a nopaline synthase (NOS) gene, a terminator of an octopine synthase (OCS) gene, a CaMV 35S terminator, a 3′ terminator of E. coli lipopolyprotein lpp, a trp operon terminator, an amyB terminator or a terminator of an ADH1 gene and the like is mentioned. The sequence is not particularly limited as long as it is a sequence capable of terminating the transcription of the gene transcribed by the promoter.

As an enhancer, e.g., an enhancer region including an upstream sequence within the CaMV 35S promoter is mentioned. The enhancer is not particularly limited so long as it can enhance the expression efficiency of nucleic acids or the like encoding active peptides.

As a selecting marker gene, a drug resistant gene (for example, a tetracycline resistant gene, an ampicillin resistant gene, a kanamycin resistant gene, a hygromycin resistant gene, a spectinomycin-resistant gene, a chloramphenicol resistant gene or a neomycin resistant gene); a fluorescent or luminescent reporter gene (for example, luciferase, β-galactosidase, β-glucuronidase (GUS) or green fluorescent protein (GFP)); or an enzyme gene such as a neomycin phosphotransferase II (NPT II) gene or a dihydrofolate reductase gene and the like is mentioned.

According to the recombinant vector of the present invention, e.g., the operation and/or expression of the polynucleotide described in the second aspect can be easily controlled. In addition, expression of CSyGT in the host cell can be operated.

### 4. Transformant or a Progeny Thereof

### 4-1. Summary

A fourth aspect of the present invention relates to a transformant or a progeny thereof. The transformant or the progeny thereof of the present invention contains the polynucleotide described in the second aspect or the recombinant vector described in the third aspect in the cell, and is capable of cloning CSyGT gene and the like, and/or expressing CSyGT and the like. According to the transformant of the present invention, CSyGT and the like can be stably biologically synthesized using in vivo expression system.

### 4-2. Configuration

In the specification, the “transformant” refers to a host transformed by introducing the polynucleotide described in the second aspect or the recombinant vector described in the third aspect.

The host to be transformed is not particularly limited. For example, bacteria such as Escherichia coli or Bacillus subtilis; yeast such as a budding yeast (Saccharomyces cerevisiae), a fission yeast (Schizosaccharomyces pombe) or methanol utilizing yeast (Pichia pastoris); fungus such as Aspergillus, Neurospora, Fuzarium or Trichoderma; a monocotyledonous plant or dicotyledonous plant; or a plant cell, a mammal cell or an insect cell (for example, sf9 or sf21) is mentioned. A Fabaceae plant or yeast is preferred.

The transformant of the present invention includes a clone having the same genetic information. For example, if a host is a single cell microbe which asexually reproduces, such as E. coli and yeast, a newly produced clone from a first-generation transformant by e.g., fissuration or budding, is also included in the transformant of the invention. If a host is a plant, a part of a plant body taken from a first-generation transformant, for example, a plant tissue such as epidermis, phloem, parenchyma, xylem or fibrovascular bundle, a plant organ such as a leaf, a petal, a stem, a root or a seed; a clone obtained from a plant cell by plant tissue culture, cuttage, grafting or layering; or a newly produced clone by asexual reproduction from a vegetative reproduction organ, which is obtained from a first-generation transformant, such as rhizome, tuberous root, corm or runner, is also included in the transformant of the invention.

The transformant of the present invention may further have one or more other polynucleotides or recombinant vectors, in addition to the polynucleotide described in the second aspect or a recombinant vector described in the third aspect. The other polynucleotides herein refer to polynucleotides except the polynucleotide described in the second aspect. For example, j-amyrin synthase gene, CYP88D6 or CYP72A154 or any one of the oleanane-type triterpenoid monoglucuronide synthase genes corresponds thereto. The other recombinant vectors refer to recombinant vectors except the recombinant vector described in the third aspect.

The transformant of the present invention can be prepared by introducing a polynucleotide or recombinant vector as mentioned above into an appropriate host.

As a method for introducing a polynucleotide or recombinant vector as mentioned above, a method known in the art such as an agrobacterium method, a PEG-calcium phosphate method, an electroporation method, a liposomal method, a particle-gun method or a microinjection method can be used. The polynucleotide introduced may be integrated into the genomic DNA of a host or may be present while keeping the state of the polynucleotide (for example, still present in a foreign vector) just introduced. Furthermore, the polynucleotide introduced may be continuously maintained in a host cell like a case where the polynucleotide is integrated into the genomic DNA of a host or may be temporarily retained.

After the polynucleotide described in the second aspect or the recombinant vector described in the third aspect is introduced into a host by the aforementioned method, whether the desired polynucleotide is introduced or not can be checked by e.g., a PCR method, a Southern hybridization method, a Northern hybridization method or an in-situ hybridization.

In the specification, the “progeny thereof” is a progeny obtained by sexual reproduction of a first-generation transformant and means a host having the polynucleotide described in the second aspect or the recombinant vector described in the third aspect of the present invention in an expressible state, and preferably a host progeny having the polynucleotide or the polynucleotide described in the second aspect in the recombinant vector in an expressible state. For example, if the transformant is a plant, a seedling of the transformant corresponds thereto. The generation of the progeny is not a matter.

According to the transformant of the present aspect, an oleanane-type triterpenoid present in a host cell can be converted into an oleanane-type triterpenoid monoglucuronide by enhancing the expression of the polynucleotide introduced. In addition, by changing the host of the transformant, glucuronosyltransferase 1 to which a different sugar chain is added can be obtained. For example, when the host of the transformant is yeast, glucuronosyltransferase 1 to which a high mannose type sugar chain is added is expressed, unlike the case where a Fabaceae plant is used as a host. It is because glycosylation reactions in yeast are different from those in plants (Strasser R. Glycobiology, 2016, 26(9): 926-939).

### 5. Method for Producing Glucuronosyltransferase 1 and Active Fragment Thereof (CSyGT, Etc.)

### 5-1. Summary

A fifth aspect of the present invention relates to a method for producing CSyGT and the like, the method comprising culturing transformant of the fourth aspect or a progeny thereof and extracting a polypeptide having glucuronic acid transfer activity 1 described in the first aspect, that is, CSyGT and the like, from a culture thereof. According to the method for producing the polynucleotide of the present invention, CSyGT and the like can be obtained stably and in a large amount by using the host as a biological production system.

### 5-2. Method

The production method of the present invention includes a culturing step and an extraction step as essential steps. Hereinafter, each step will be specifically described.

### (1) Culturing Step

In the present aspect, “culturing step” is a step of culturing a transformant of the fourth aspect or a progeny thereof. As for the transformant or the progeny thereof used in the present invention, it is preferable to use a transformant that can over-express or constitutively express the polypeptide described in the first aspect or a progeny thereof. For example, in a case of a transformant having the recombinant vector described in the third aspect or a progeny thereof, it is preferable that the recombinant vector is an expression vector containing an over-expression promoter or a constitutive promoter. As for the transformant of the fourth aspect or a progeny thereof, any host may be acceptable, but is preferably a Fabaceae plant or yeast. By changing the host, it is possible to obtain glycoproteins to which a different sugar chain is added, even if the same glucuronosyltransferase 1 described in the first step is expressed.

As a medium for culturing, a medium suitable for culturing a host may be appropriately used. As the medium, a medium known in the art can be used. Although the medium is not limited, if culture is made by using bacteria such as E. coli as a host, for example, LB medium or M9 medium and the like is mentioned. If culture is made by using a yeast as a host, YPD medium, YPG medium, YPM medium, YPDM medium or SMM medium and the like, is mentioned. If culture is made by using a plant as a host, an appropriate culture soil or a hydroponic culture medium and the like is mentioned.

The medium may appropriately contain, for example, a carbon source (e.g., glucose, glycerin, mannitol, fructose, lactose), a nitrogen source (e.g., an inorganic nitrogen source such as ammonium sulfate, ammonium chloride; an organic nitrogen source such as a casein digest, a yeast extract, polypeptone, BACTO tryptone, a beef extract), an inorganic salt (e.g., sodium diphosphate, potassium diphosphate, magnesium chloride, magnesium sulfate, calcium chloride), a vitamin (e.g., vitamin B1), and a drug (an antibiotic substance such as ampicillin, tetracycline, kanamycin).

The culture conditions are not particularly limited as long as they are appropriate conditions for expressing a polynucleotide. A culture is performed usually at a temperature of 10 to 45 °C, 15 to 40 °C or 18 to 37 °C, if necessary, while aerating, under irradiation, and/or with stirring for several hours to several hundreds of hours.

### (2) Extraction Step

In the present aspect, “extraction step” refers to a step of extracting CSyGT and the like from the culture obtained in the culturing step.

In the specification, “culture” refers to a culture supernatant or a cultured transformant. The CSyGT and the like secreted from the transformant may be contained not only in the cells of the transformant but also in the content supernatant.

To recover the polypeptide described in the first aspect from a culture, the polypeptide present in the culture is extracted by a known method and purified, as needed. For example, a desired polypeptide can be obtained by a solvent extraction method, a salting-out method, a solvent precipitation method, a dialysis method, an ultrafiltration method, a gel electrophoresis method, a gel filtration chromatography, ion exchange chromatography, reversed phase chromatography and affinity chromatography and the like, singly or in appropriate combination. Specifically, see the method of Hayashi et al. (Hayashi et al., 1996, Phytochemistry, 42: 665-666) and the method of Noguchi et al. (Noguchi et al., 2007, J. Biol. Chem., 282: 23581-23590) mentioned above. It is also possible to recover the desired polypeptide described in the first aspect of interest based on a host-specific sugar chain. For example, when the transformant of the fourth aspect or a progeny thereof is yeast, the expressed polypeptide of the present invention has a high mannose type sugar chain added thereto. Therefore, the polypeptide can also be extracted and purified using mannose-binding lectin (for example, UDA lectin, BC2L-A lectin, etc.).

### 6. Gene Recombinant for Producing Glycyrrhizin

### 6-1. Summary

The sixth aspect of the present invention is a gene recombinant for producing glycyrrhizin. The gene recombinant contains an expression vector that expresses one set of enzymes required for the biosynthetic pathway from β-amyrin to glycyrrhizin in Glycyrrhiza plants, i.e., a set of four enzymes that catalyze a two-step oxidation reaction and a two-step glycosylation reaction. The gene recombinant of the invention can biologically synthesize glycyrrhizin from β-amyrin in organism cells, and thus can be used as a biological production system for glycyrrhizin.

### 6-2. Configuration

### 6-2-1. Included Expression Vector

The gene recombinant of the present invention characteristically contains at least an expression vector that contains a polynucleotide encoding a set of four enzymes and/or an active fragment thereof required for the biosynthetic pathway from β-amyrin to glycyrrhizin in a host cell. If necessary, it may further contain an expression vector containing a polynucleotide encoding a β-amyrin synthase gene. The four enzymes are polypeptides that catalyze respectively the first-step oxidation reaction and the second-step oxidation reaction of β-amyrin, the first-step glycosylation reaction and the second-step glycosylation reaction of oleanane-type triterpenoids. Expression vectors containing each enzyme or an active fragment thereof are shown in (1) to (4) below and will be specifically described. In (1) through (4), each of the four expression vectors is described separately, but the gene for each enzyme may be contained in different expression vectors, or two or more of them may be contained in the same expression vector.

### (1) CYP88D6 Expression Vector

“CYP88D6 expression vector” contains a gene encoding a polypeptide having an activity of oxidizing the 11-position in an oleanane-type triterpenoid, that is, CYP88D6 and an active fragment thereof (often referred to herein as “CYP88D6 and the like”) and a fragment thereof (often referred to herein as “CYP88D6 gene and the like”). Therefore, CYP88D6 and the like are expressed by the CYP88D6 expression vectors in the gene recombinant.

Specific examples of the CYP88D6 include, but are not limited to, CYP88D6 derived from Glycyrrhiza (G. uralensis) consisting of the amino acid sequence represented by SEQ ID NO: 7. Further, a polypeptide having the first-step oxidation activity and consisting of the amino acid sequence derived from the amino acid sequence represented by SEQ ID NO: 7 by deletion, replacement or addition of one or a plurality of amino acids, or consisting of the amino acid sequence having 80% or more identity with the amino acid sequence represented by SEQ ID NO: 7, is also illustrated.

Without limitation, in the gene recombinant of the present invention, 11-oxo-p-amyrin can be produced by mainly using endogenous or exogenous β-amyrin as a substrate and oxidizing the 11-position thereof by a catalytic activity of CYP88D6 and the like expressed by the CYP88D6 expression vector. Alternatively, 30-hydroxy-11-oxo-β-amyrin can be produced by using 30-hydroxy-β-amyrin as a substrate and oxidizing the 11-position thereof. Further alternatively, glycyrrhetinic acid can be produced by using 11-deoxoglycyrrhetinic acid as a substrate and oxidizing the 11-position thereof.

The configuration of the plasmid region in the CYP88D6 expression vector is equivalent to that of the expression vector in the recombinant vector described in the third aspect. The recombinant vector described in Japanese Patent No. 5526323 may also be used.

### (2) CYP72A154 Expression Vector

“CYP72A154 expression vector” contains a gene encoding a polypeptide having an activity of oxidizing the 30-position in an oleanane-type triterpenoid, that is, CYP72A154 and an active fragment thereof (often referred to herein as “CYP72A154 and the like”) and a fragment thereof (often referred to herein as “CYP72A154 gene and the like”). Therefore, CYP72A154 and the like are expressed by the CYP72A154 expression vectors in the gene recombinant.

Specific examples of the CYP72A154 include, but are not limited to, Glycyrrhiza (G. uralensis)-derived CYP72A154 consisting of the amino acid sequence represented by SEQ ID NO: 9, Glycyrrhiza glabra (G. glabra)-derived CYP72A154 consisting of the amino acid sequence represented by SEQ ID NO: 11, and M. truncatula (Medicago truncatula)-derived CYP72A63 consisting of the amino acid sequence represented by SEQ ID NO: 13. Further, a polypeptide having the second-step oxidation activity and consisting of the amino acid sequence derived from the amino acid sequence represented by any one of SEQ ID NOs: 9, 11, and 13 by deletion, replacement or addition of one or a plurality of amino acids, or consisting of the amino acid sequence having 80% or more identity with the amino acid sequence represented by any one of SEQ ID NOs: 9, 11, and 13, is also illustrated.

Without limitation, in the gene recombinant of the present invention, 30-hydroxy-B-amyrin and 30-hydroxy-11-oxo-β-amyrin can be produced, respectively, mainly using (-amyrin and 11-oxo-β-amyrin as substrates and oxidizing the 30-position by a catalytic activity of CYP72A154 and the like expressed by the CYP72A154 expression vector. In addition, glycyrrhetinic acid can be produced by using 30-hydroxy-11-oxo-β-amyrin as a substrate and further oxidizing the 30-position thereof.

The configuration of the plasmid region in the CYP72A154 expression vector is equivalent to that of the expression vector in the recombinant vector described in the third aspect. The recombinant vector described in Japanese Patent No. 5771846 may also be used.

### (3) UGT73P12 Recombinant Vector

The “UGT73P12 recombinant vector” contains a gene encoding a polypeptide having an activity of transferring glucuronic acid to the hydroxy group at the 2-position of the glucuronic acid in an oleanane-type triterpenoid monoglucuronide, that is, UGT73P12 and an active fragment thereof (often referred to herein as “UGT73P12 and the like”) and a fragment thereof (often referred to herein as “UGT73P12 gene and the like”). Therefore, in the gene recombinant, UGT73P12 and the like are expressed by the UGT73P12 expression vectors.

Specific examples of the UGT73P12 include, but are not limited to, UGT73P12 derived from Glycyrrhiza (G. uralensis) consisting of the amino acid sequence represented by SEQ ID NO: 15. Further, a polypeptide having the second-step glycosylation activity and consisting of the amino acid sequence derived from the amino acid sequence represented by SEQ ID NO: 15 by deletion, replacement or addition of one or a plurality of amino acids, or consisting of an amino acid sequence having 80% or more identity with the amino acid sequence represented by SEQ ID NO: 15, is also illustrated.

The configuration of the plasmid region in the UGT73P12 expression vector is equivalent to that of the expression vector in the recombinant vector described in the third aspect. The recombinant vector described in Japanese Patent No. 6344774 may also be used.

### (4) CSyGT Expression Vector

Since the “CSyGT expression vector” corresponds to the CSyGT expression vector in the CSyGT recombinant vector described in the third aspect, detailed description thereof will be omitted here.

### 6-2-2. Gene Recombinant for Producing Glycyrrhizin

The “gene recombinant for producing glycyrrhetinic acid” of the present invention refers to a transformant into which an expression vector containing at least the set of four enzyme genes is introduced, or a progeny thereof having the set of enzyme genes. Therefore, the basic configuration may be the same as that of the transformant described in the fourth aspect and the progeny thereof, except that the types of the expression vectors included are different. However, since the present invention is a gene recombinant capable of biologically synthesizing glycyrrhizin from β-amyrin in the cell, it is preferably a host capable of biologically synthesizing β-amyrin, which is the starting material in the biosynthetic system, in the cell. The biosynthesis of β-amyrin in the host may be based on an endogenous synthetic system, or on an exogenous synthetic system. Since β-amyrin, which is an oleanane-type triterpenoid, can be biologically synthesized by many plants, the host of the present invention is preferably a plant when based on an endogenous synthetic system. Plant species with high β-amyrin synthesis capacity, high fertility, and easy to grow, are preferred. A plant relatively related to Glycyrrhiza, that is, a Fabaceae plant is more preferred. For example, a species belonging to the genus Glycyrrhiza, a species belonging to the genus Soybean, a species belonging to the genus Lotus japonicus and the like may be included. On the other hand, when the biosynthesis of β-amyrin is based on an exogenous synthetic system, the host itself may be a species that cannot biologically synthesize β-amyrin. For example, by introducing an expression vector containing a β-amyrin synthase gene into an yeast, a transformant of the yeast can be used as a host capable of biologically synthesizing β-amyrin in the cell.

According to the present invention, even a host that could not biologically synthesize glycyrrhizin in the past, it becomes able to biologically synthesize glycyrrhizin as a metabolite of β-amyrin, which is used as a starting material.

### 7. Method for Producing Glycyrrhizin

### 7-1. Summary

The seventh aspect of the present invention is a method for producing glycyrrhizin. The production method of the present invention is characterized in that glycyrrhizin is produced from β-amyrin by using the gene recombinant for producing glycyrrhizin of the sixth aspect as a biological production system.

According to the production method of the present invention, glycyrrhizin, which was expensive in the past, can be stably obtained and in a large amount without extraction from Glycyrrhiza.

### 7-2. Method

The production method of the present invention comprises a culturing step as an essential step, and “extraction step” as an optional step.

### (1) Culturing Step

The “culturing step” in the present aspect may be basically equivalent to the culture step described in the fifth aspect. When the gene recombinant is a plant, a method for culturing a plant under known conditions may be applied. According to the present step, glycyrrhizin is produced in the gene recombinant for producing glycyrrhizin of the sixth aspect.

### (2) Extraction Step

“Extraction step” in this aspect may be basically equivalent to the extraction step described in the fifth aspect. If the gene recombinant is a plant, the same method as that for extracting glycyrrhizin from Glycyrrhiza can be used.

According to the production method of the present invention, glycyrrhizin can be obtained stably and in a large amount from various gene recombinants, without extraction from Glycyrrhiza.

### EXAMPLES

The present invention will be described by way of examples below.

### Example 1: Isolation of Soybean-Derived Cellulose Synthase Analogous Gene Glyma.06G324300

Matured seeds of greenhouse-grown soybean (Glycine max) “Williams 82” variety were collected. Total RNA was prepared by using an RNA extraction reagent, RNeasy Plant Mini Kit (QIAGEN) in accordance with the attached protocol. Using the total RNA (200 ng) obtained, a first-strand cDNA was synthesized by use of QuantiTech Reverse Transcription Kit (QIAGEN) in accordance with the attached protocol. A cycle of PCR was repeated 30 times by use of PrimeSTAR GXL DNA Polymerase (TAKARA BIO INC.) at an annealing temperature of 55 °C and a reaction temperature of 68 °C, using the 5-fold diluted first-strand cDNA (1 μL for each) as a template and oligo DNA molecules corresponding to the N terminal and C terminal of the polypeptide estimated from Glyma.06G324300, respectively, as a forward primer (SEQ ID NO: 17) and a reverse primer (SEQ ID NO: 18). Note that 12 nucleotides (AAAAAGCAGGCT; SEQ ID NO: 51) are artificially added to the 5′ terminal of the forward primer and 12 nucleotides (AGAAAGCTGGGT; SEQ ID NO: 52) are artificially added to the 5′ terminal of the reverse primer, for the reason that the nucleotides are required for nucleotide sequence specific recombination reaction (GATEWAY attB×attP reaction) in cloning into pDONR™221 (Thermo Fisher Technologies). The DNA fragment, which was amplified from seed-derived first-strand cDNA, was cloned to pDONR™221 by the nucleotide sequence specific recombination reaction (GATEWAY attB×attP reaction) using Gateway BP Clonase II Enzyme Mix (Thermo Fisher Technologies). The polynucleotide sequences of the resultant three independent clones were determined. The sequence thus obtained is represented by SEQ ID NO: 2. The polypeptide sequence estimated from the aforementioned sequence is represented by SEQ ID NO: 1.

### Example 2: Search for Glycyrrhiza-Derived Glyma.06G324300 Homologous Gene

Glyma.06G324300 homologous gene was searched as a candidate for Glyma.06G324300 orthologue gene by gene homology search from Glycyrrhiza (Glycyrrhiza uralensis), which belongs to the same Fabaceae plant as soybean and is known to biologically synthesize glycyrrhizin. Using BLAST homology search function in genome information database of Glycyrrhiza, i.e., Glycyrrhiza uralensis GDB (http-colon-slash-slash ngs-data-archive.psc.riken.jp/Gur-genome/index.pl), a type of nucleotide sequence Glyur003152s00037491, which may encode a protein having high amino acid identity with Glyma.06G324300, was found. The polypeptide estimated from Glyur003152s00037491 had an amino acid identity of 81% with that of Glyma.06G324300.

### Example 3: Isolation of Glycyrrhiza-Derived Glyma.06G324300 Homologous Gene

Total RNA was prepared from root of Glycyrrhiza using RNA extraction reagent, PureLink Plant RNA Reagent (Thermo Fisher Scientific). Using the total RNA (1 μg) obtained, a first-strand cDNA was synthesized by use of SMART RACE cDNA amplification kit (Clontech) in accordance with the attached protocol. A cycle of PCR was repeated 30 times by use of PrimeSTAR Max DNA Polymerase (TAKARA BIO INC.) at an annealing temperature of 55 °C and a reaction temperature of 72 °C, using the first-strand cDNA (2 μL) as a template and oligo DNA molecules corresponding to the N terminal and C terminal of the polypeptide estimated from Glyur003152s00037491, respectively, as a forward primer (SEQ ID NO: 19) and a reverse primer (SEQ ID NO: 20). Note that 4 nucleotides (cacc) are artificially added to the 5′ terminal of the forward primer, for the reason that the nucleotides are required for cloning into pENTR™/D-TOPO (registered trademark) entry vector (Thermo Fisher Technologies). The DNA fragment, which was amplified, was cloned to pENTR™/D-TOPO entry vector. The nucleotide sequences of the resultant four independent clones were determined. As a result, the nucleotide sequence of Glyma.06G324300 homologous gene of Glycyrrhiza thus obtained is represented by SEQ ID NO: 4. The polypeptide sequence estimated from the nucleotide sequence is represented by SEQ ID NO: 3. The amino acid sequence represented by SEQ ID NO: 3 had an identity of 82% with the amino acid sequence represented by SEQ ID NO: 1.

### Example 4: Search for Lotus japonicus-Derived Glyma.06G324300 Homologous Gene

In the same manner as in Example 2, Glyma.06G324300 homologous gene was searched as a candidate for a Lotus japonicus-derived Glyma.06G324300 orthologue gene. Using BLAST homology search function in genome information database of L. japonicus, i.e., miyakogusa.jp (http-colon-slash-slash www.kazusa.or.jp/lotus/release1/index.html), a type of nucleotide sequence, Lj3g3v1981230, which may encode a protein having high amino acid identity with Glyma.06G324300, were found. The polypeptide estimated from Lj3g3v1981230 had an amino acid identity of 81.4% with that of Glyma.06G324300.

### Example 5: Isolation of Lotus japonicus-Derived Glyma.06G324300 Homologous Gene

Using the total RNA (1 μg) obtained from Lotus japonicus, a first-strand cDNA was synthesized by use of SMART RACE cDNA amplification kit (Clontech) in accordance with the attached protocol. A cycle of PCR was repeated 30 times by use of PrimeSTAR Max DNA Polymerase (TAKARA BIO INC.) at an annealing temperature of 55 °C and a reaction temperature of 72 °C, using the first-strand cDNA (2 μL) as a template and oligo DNA molecules corresponding to the N terminal and C terminal of the polypeptide estimated from Lj3g3v1981230, respectively, as a forward primer (SEQ ID NO: 37) and a reverse primer (SEQ ID NO: 38). Note that 4 nucleotides (cacc) are artificially added to the 5′ terminal of the forward primer, for the reason that the nucleotides are required for cloning into pENTR™/D-TOPO (registered trademark) entry vector (Thermo Fisher Technologies). The DNA fragment, which was amplified, was cloned to pENTR™/D-TOPO entry vector. The polynucleotide sequences of the resultant two independent clones were determined. As a result, the nucleotide sequence of Glyma.06G324300 homologous gene of Lotus japonicus thus obtained is represented by SEQ ID NO: 6. The polypeptide sequence estimated from the nucleotide sequence of SEQ ID NO: 6 is represented by SEQ ID NO: 5. The amino acid sequence represented by SEQ ID NO: 5 had an identity of 82% with the amino acid sequence represented by SEQ ID NO: 1.

### Example 6: Construction of Destination Vector for Yeast Expression

In order to investigate the expected transglycosylation activity of Glyma.06G324300 isolated in Examples 1, 3, and 5 and a homologous protein thereof, an expression vector for each protein was constructed using a yeast expression system.

The yeast (Saccharomyces cerevisiae) INVSc1 strain used does not contain endogenous UDP-glucuronic acid, which is to be a glycosyl donor substrate, in the glycosylation reaction expected for the candidate gene product. Therefore, a UDP-glucose dehydrogenase (UGD) gene, which synthesizes UDP-glucuronic acid using UDP-glucose endogenously in yeast as a substrate, was introduced into a destination vector for yeast expression. Specifically, a cycle of PCR was repeated 30 times by use of PrimeSTAR Max DNA Polymerase (TAKARA BIO INC.) at an annealing temperature of 55 °C and a reaction temperature of 72 °C, using cDNA of Arabidopsis thaliana-derived UGD(AtUGD2) as a template and oligo DNA molecules corresponding to the N terminal and C terminal of the polypeptide, respectively, as a forward primer (SEQ ID NO: 21) and a reverse primer (SEQ ID NO: 22). Note that 15 nucleotides upstream of the cloning position of the destination vector and 4 nucleotides (aaaa), 19 nucleotides in total, are artificially added to the 5′ terminal of the polynucleotide represented by SEQ ID NO: 23 (gggcggccgcactag) of the forward primer, for the reason that the nucleotides are required for in-fusion cloning. Further, note that 15 nucleotides downstream of the cloning position of the destination vector are added to the 3′ terminal of the polynucleotide represented by SEQ ID NO: 24 (atccatcgatactag) of the reverse primer. Destination vector pESC-HIS-GW was produced by introducing Gateway cassette A (Thermo Fisher Technologies) at the Srff restriction enzyme site in MCS2 of pESC-HIS (registered trademark) yeast expression vector (Agilent Technologies). The Destination vector pESC-HIS-GW was treated with SpeI restriction enzyme, mixed with DNA fragments amplified from cDNA, and the DNA fragment represented by SEQ ID NO: 25 is introduced at MCS1 in pESC-HIS-GW using In-Fusion (registered trademark) HD Cloning Kit (TAKARA BIO INC.), to obtain the destination vector pESC-HIS-AtUGD2-GW.

### Example 7: Construction of Yeast Expression Clone

A plasmid (entry clone) having the polynucleotide represented by SEQ ID NO: 2 produced in Example 1 and a destination vector pESC-HIS-AtUGD2-GW produced in Example 6 were mixed with each other and subjected to a nucleotide sequence specific recombination reaction (GATEWAY attL×attR reaction) using Gateway LR ClonaseII Enzyme Mix (Thermo Fisher Technologies) to transfer the DNA fragment represented by SEQ ID NO: 2 to pESC-HIS. In this manner, a yeast expression vector pESC-HIS-AtUGD2-Glyma.06G324300 for a gene represented by SEQ ID NO: 2 was obtained. In the same manner as described above, yeast expression vectors pESC-HIS-AtUGD2-Glyur003152s00037491 and pESC-HIS-AtUGD2-Lj3g3v1981230, respectively for the genes represented by SEQ ID NOs: 4 and 6 produced in Examples 3 and 5, were respectively obtained.

### Example 8: Introduction into Glycyrrhetinic Acid and Soyasapogenol B-Producing Yeast Strains

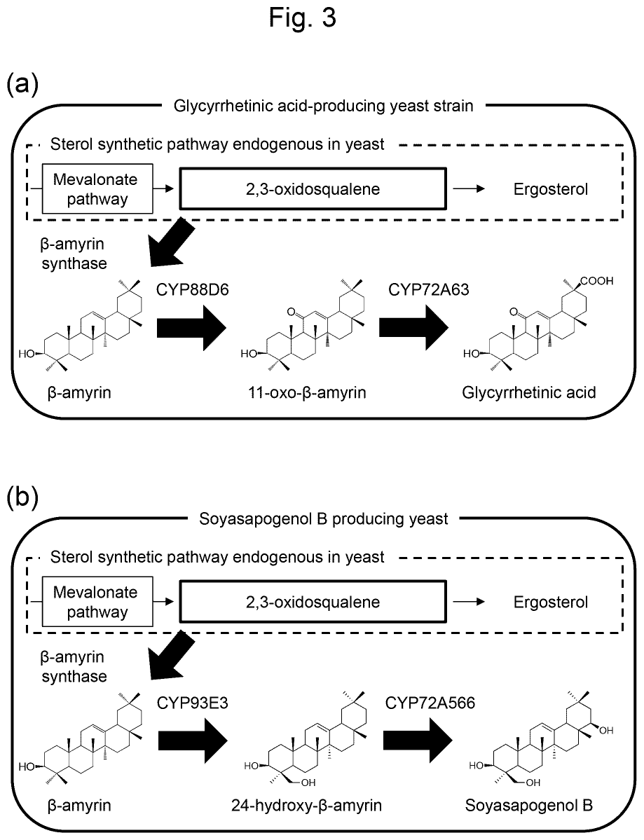

To yeast INVScI strain (Thermo Fisher Technologies) (MATa his3D1 leu2 trp1-289 ura3-52 MATAlpha his3D1 leu2 trp1-289 ura3-52), the expression vector pYES3-BAS of the β-amyrin synthase (LjOSC1) gene of Lotus japonicus, the co-expression vector pESC-CPR-CYP88D6 of the CYP88D6 gene and the cytochrome P450 reductase (LjCPR1) of Lotus japonicus, and the expression vector pDEST52-CYP72A63 of the CYP72A63 gene which is the M. truncatula ortholog of the Glycyrrhiza CYP72A154 gene were introduced, and co-expressed, to obtain a glycyrrhetinic acid-producing yeast strain (FIG. 3(a)). At the same time, the expression vector pYES3-BAS of the β-amyrin synthase gene, the co-expression vector pESC-CPR-CYP93E3 of the CYP93E3 gene and the cytochrome P450 reductase (LjCPR1) of Lotus japonicus, and the expression vector pDEST52-CYP72A566 of the CYP72A566 gene were introduced, and co-expressed, to obtain soyasapogenol B-producing yeast (FIG. 3(b)). pESC-HIS-AtUGD2-Glyma.06G324300, pESC-HIS-AtUGD2-Glyur003152s00037491, and pESC-HIS-AtUGD2-Lj3g3v1981230, obtained in Example 7, were respectively introduced into these yeast strains. As a negative control, pESC-HIS-AtUGD2 corresponding to an empty vector was introduced into the glycyrrhetinic acid-producing yeast strain. The yeast was transformed by use of Frozen-EZ Yeast Transformation II (Zymo Research) in accordance with the attached protocol.

### Example 9: In Vivo Enzyme Assay Using Recombinant Yeast

Using 1 mL of Yeast nitrogen base (YNB) medium (-Trp/-Leu/-Ura/-His) containing 2% glucose, a glycyrrhetinic acid-producing yeast strain having pESC-HIS-AtUGD2-Glyma.06G324300, pESC-HIS-AtUGD2-Glyur003152s00037491, pESC-HIS-AtUGD2-Lj3g3v1981230, or negative control pESC-HIS-AtUGD2, obtained in Example 8, was cultured while shaking at 30 °C, 200 rpm for 24 hours. Thereafter, the culture solution was centrifuged at 3,000 g, 4 °C for 5 minutes to obtain a yeast cell pellet. The yeast cell pellet thus obtained was suspended in 1 mL of Yeast nitrogen base (YNB) medium (-Trp/-Leu/-Ura/-His), thereafter centrifuged again at 3,000 g, 4 °C for 5 minutes to obtain a yeast cell pellet. The yeast cell pellet thus obtained was suspended in 5 mL of Yeast nitrogen base (YNB) medium (-Trp/-Leu/-Ura/-His) containing 2% galactose, and cultured while shaking at 30 °C, 200 rpm for 5 days. Thereafter, a volume equivalent to 1 mL of glass beads (SIGMA) and 4 mL of 1-butanol were added to the culture solution. In order to fracture yeast cells, the culture solution was vigorously stirred with a strong shaker for 30 minutes, and the resultant solution was centrifuged at 10,000 g, 4 °C for 10 minutes, thereafter the supernatant was recovered as a yeast metabolite extract. To the remaining solution, 4 mL of 1-butanol was added newly and stirred again for 30 minutes, the resultant solution was centrifuged at 10,000 g, 4 °C for 10 minutes, and the supernatant was extracted. As a result, a metabolite extract derived from the glycyrrhetinic acid-producing yeast strain and expressing the polypeptide represented by SEQ ID NO: 1 (Glyma.06G324300) (sample A), a metabolite extract derived from the glycyrrhetinic acid-producing yeast strain and expressing the polypeptide represented by SEQ ID NO: 3 (Glyur003152s00037491) (sample B), a metabolite extract derived from the glycyrrhetinic acid-producing yeast strain and expressing the polypeptide represented by SEQ ID NO: 5 (Lj3g3v1981230) (sample C), and a metabolite extract derived from the glycyrrhetinic acid-producing yeast strain with an empty vector alone and expressing no gene (sample D) were obtained.

In the same manner, the soyasapogenol B-producing yeast strain having pESC-HIS-AtUGD2-Glyma.06G324300, pESC-HIS-AtUGD2-Glyur003152s00037491, pESC-HIS-AtUGD2-Lj3g3v1981230, or negative control pESC-HIS-AtUGD2 was also cultured, and the metabolite was extracted. As a result, a metabolite extract derived from the soyasapogenol B-producing yeast strain expressing the polypeptide represented by SEQ ID NO: 1 (Glyma.06G324300) (sample E), a metabolite extract derived from the soyasapogenol B-producing yeast strain expressing the polypeptide represented by SEQ ID NO: 3 (Glyur003152s00037491) (sample F), a metabolite extract derived from the soyasapogenol B-producing yeast strain expressing the polypeptide represented by SEQ ID NO: 5 (Lj3g3v1981230) (sample G), and a metabolite extract derived from the soyasapogenol B-producing yeast strain with an empty vector alone and expressing no gene (sample H) were obtained.

### Example 10: Analysis of Yeast Metabolite Extract

Samples A, B, C, and D, as well as samples E, F, G, and H, obtained in Example 9, were evaporated using a rotary evaporator. The precipitates were suspended in 300 μL of methanol and filtered using Millex-GV, 0.22 μm, PVDF, 4 mm (Merck & Co., Inc.), and used as samples for LC-MS analysis.

LC-MS analysis was performed by ACQUITY UPLC/TQD-MS (Waters Corp.). As a column, UPLC HSS C18 (2.1 mm×150 mm, 1.7 μm) (Waters Corp.) was used. Analysis was made while supplying solvents containing 0.1% acetic acid-acetonitrile: 0.1% acetic acid-water=30: 70 (0 to 5 minutes), 40:60 to 100:0 (5 to 28 minutes), and 100:0 (28 to 31.5 minutes) at a flow rate of 0.2 mL/minute. MS was analyzed using SIM mode with the following parameters: m/z of each compound, glycyrrhetinic acid=469.7, glycyrrhetinic acid monoglycoside=631.9, glycyrrhetinic acid monoglucuronide=645.8, glycyrrhizin=821.9. The metabolite was identified by comparing LC retention times and MS spectra using samples prepared by dissolving commercially available glycyrrhetinic acid monoglucuronide and glycyrrhizin in methanol to a concentration of 1 μM as standards.

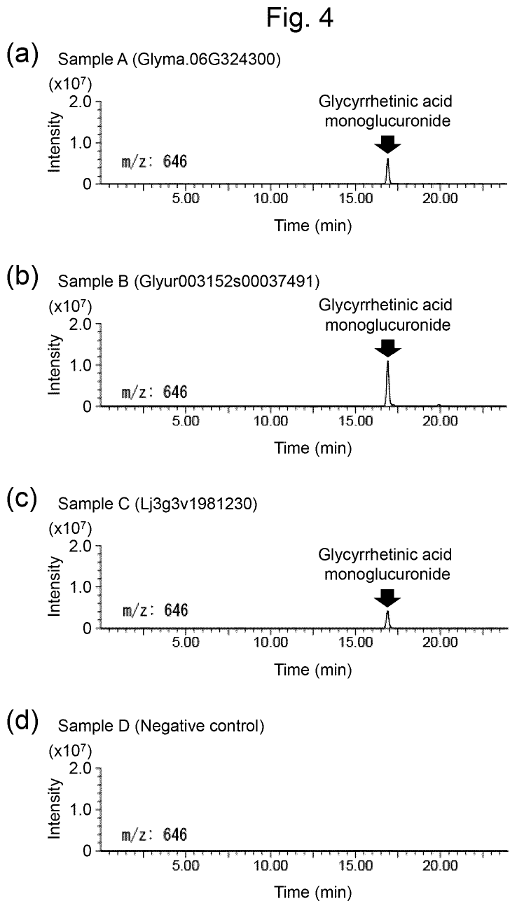

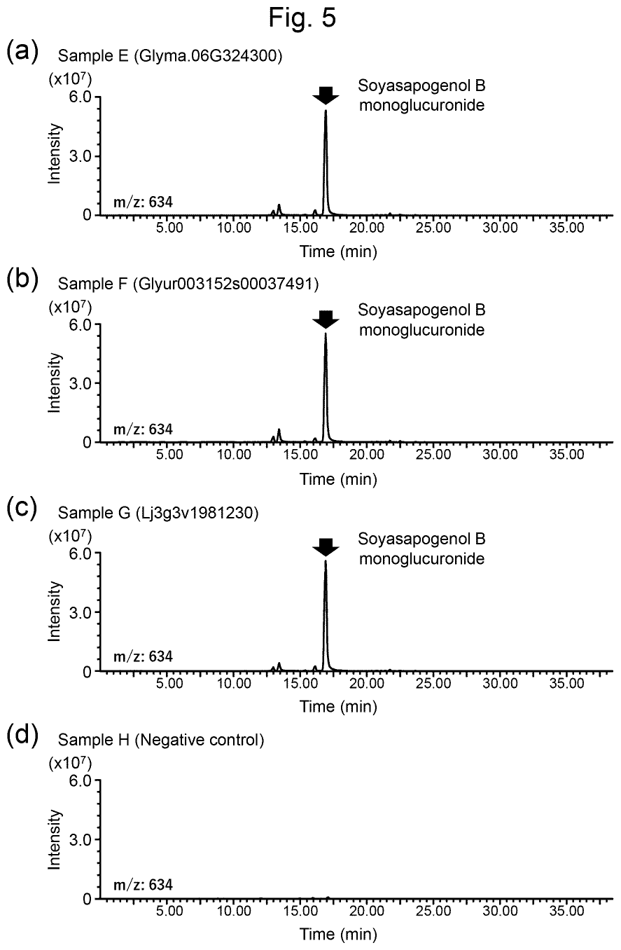

The results are shown in FIG. 4 and FIG. 5.

FIG. 4 shows the results of the enzymatic activity of Glyma.06G324300 or homologues thereof when glycyrrhetinic acid and glucuronic acid were used as substrates. A peak (solid arrow) corresponding to glycyrrhetinic acid monoglucuronide was detected from sample A in (a). The retention time and mass spectrum of this peak satisfactorily coincided with those of glycyrrhetinic acid monoglucuronide. Similarly, a peak (solid arrow) corresponding to glycyrrhetinic acid monoglucuronide was detected from sample B in (b) and sample C in (c). The retention time and mass spectrum of each peak satisfactorily coincided with those of glycyrrhetinic acid monoglucuronide. In contrast, from sample D in (d) which is a negative control, a peak corresponding to glycyrrhetinic acid monoglucuronide was not detected.

FIG. 5 shows the results of the enzymatic activity of Glyma.06G324300 or homologues thereof when soyasapogenol B and glucuronic acid were used as substrates. A peak (solid arrow) corresponding to soyasapogenol B monoglucuronide was detected from sample E in (a). A peak (solid arrow) corresponding to soyasapogenol B monoglucuronide was also detected from sample F in (b) and sample G in (c). The retention time and mass spectrum of each peak satisfactorily coincided with those of soyasapogenol B monoglucuronide. In contrast, from sample H in (d) which is a negative control, a peak corresponding to soyasapogenol B monoglucuronide was not detected.

### Example 11: Preparation of Transformed Yeast for Substrate Feeding Assay

pESC-HIS-AtUGD2-Glyma.06G324300, pESC-HIS-AtUGD2-Glyur003152s00037491, and pESC-HIS-AtUGD2-Lj3g3v1981230, obtained in Example 8, were respectively introduced into the yeast INVScI strains. As a negative control, pESC—HIS-AtUGD2 corresponding to an empty vector was introduced into the same yeast INVScI strain. The yeast was transformed by use of Frozen-EZ Yeast Transformation II (Zymo Research) in accordance with the attached protocol.

### Example 12: Substrate Feeding Assay Using Recombinant Yeast

Using 2 mL of Yeast nitrogen base (YNB) medium (-His) containing 2% glucose, a transformed yeast having pESC-HIS-AtUGD2-Glyma.06G324300, pESC-HIS-AtUGD2-Glyur003152s00037491, pESC-HIS-AtUGD2-Lj3g3v1981230, or negative control pESC—HIS-AtUGD2, obtained in Example 11, was cultured while shaking at 30 °C, 200 rpm for 24 hours. Thereafter, the culture solution was centrifuged at 3,000 g, 4 °C for 5 minutes to obtain a yeast cell pellet. The yeast cell pellet thus obtained was suspended in 2 mL of Yeast nitrogen base (YNB) medium (-His), thereafter centrifuged again at 3,000 g, 4 °C for 5 minutes to obtain a yeast cell pellet. The yeast cell pellet thus obtained was suspended in 10 mL of Yeast nitrogen base (YNB) medium (-His) containing 2% galactose, and divided in equal halves, 5 mL each. Glycyrrhetinic acid at a final concentration of 5 μM was added to one sample, and soyasapogenol B at a final concentration of 5 μM was added to the other sample (FIG. 4). Thereafter, they were cultured while shaking at 30 °C, 200 rpm for 10 days. A volume equivalent to 1 mL of glass beads (SIGMA) and 4 mL of 1-butanol were added to the culture solution. In order to fracture yeast cells, the culture solution was vigorously stirred with a strong shaker for 30 minutes to fracture yeast cells, the resultant solution was centrifuged at 10,000 g, 4 °C for 10 minutes, and then the supernatant was recovered as a yeast feeding assay extract. To the remaining solution, 4 mL of 1-butanol was added newly and extracted again. As a result, a feeding assay extract prepared by adding glycyrrhetinic acid to the transformed yeast expressing the polypeptide represented by SEQ ID NO: 1 (Glyma.06G324300) (sample I), a feeding assay extract prepared by adding soyasapogenol B to the transformed yeast expressing the polypeptide represented by SEQ ID NO: 1 (Glyma.06G324300) (sample M), a feeding assay extract prepared by adding glycyrrhetinic acid to the transformed yeast expressing the polypeptide represented by SEQ ID NO: 3 (Glyur003152s00037491) (sample J), a feeding assay extract prepared by adding soyasapogenol B to the transformed yeast expressing the polypeptide represented by SEQ ID NO: 3 (Glyur003152s00037491) (sample N), a feeding assay extract prepared by adding glycyrrhetinic acid to the transformed yeast expressing the polypeptide represented by SEQ ID NO: 5 (Lj3g3v1981230) (sample K), a feeding assay extract prepared by adding soyasapogenol B to the transformed yeast expressing the polypeptide represented by SEQ ID NO: 5 (Lj3g3v1981230) (sample O), a feeding assay extract prepared by adding glycyrrhetinic acid to the transformed yeast with an empty vector alone and expressing no gene (sample L), and a feeding assay extract prepared by adding soyasapogenol B to the transformed yeast with an empty vector alone and expressing no gene (sample P) were obtained.

### Example 13: Analysis of Substrate Feeding Assay Extract

Samples I, J, K, L, M, N, O, and P obtained in Example 12 were evaporated using a rotary evaporator. The precipitates were suspended in 300 μL of methanol and filtered using Millex-GV, 0.22 μm, PVDF, 4 mm (Merck & Co., Inc.), and used as samples for LC-MS analysis.

LC-MS analysis was analyzed in the same manner as in Example 10, using SIM mode for MS, with the following parameters: for sample I, J, K, and L, m/z of each compound, glycyrrhetinic acid=469.7, glycyrrhetinic acid monoglycoside=631.9, glycyrrhetinic acid monoglucuronide=645.8, glycyrrhizin=821.9; for sample M, N, O, and P, m/z of each compound, soyasapogenol B=457.8, soyasapogenol B monoglycoside=619.8, soyasapogenol B monoglucuronide=633.8, soyasapogenol B diglucuronide=809.9. The metabolite was identified by comparing LC retention times and MS spectra using samples prepared by dissolving commercially available glycyrrhetinic acid monoglucuronide, glycyrrhizin, and soyasapogenol B monoglucuronide in methanol to a concentration of 1 μM as standards.

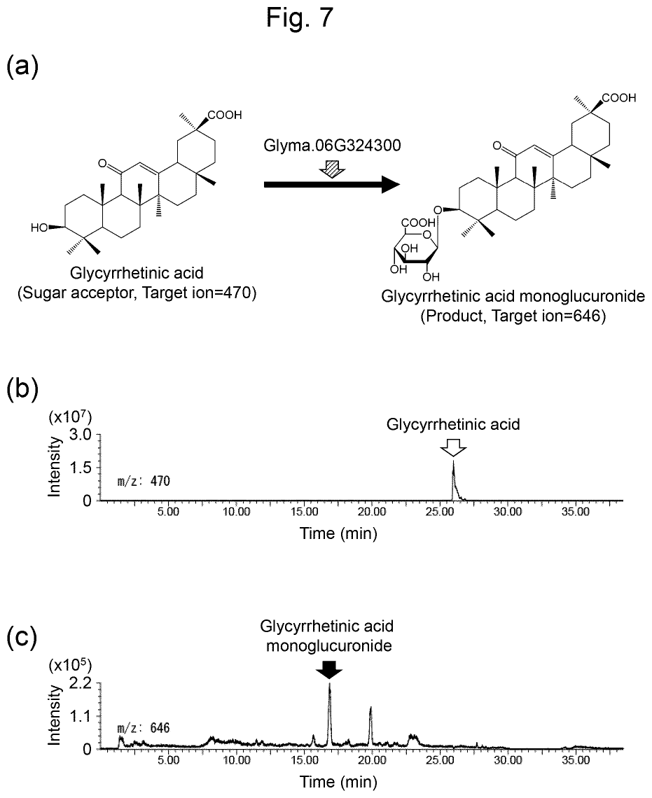

The results of using glycyrrhetinic acid as a sugar acceptor substrate are shown in FIG. 7 to FIG. 10. FIG. 7 shows results of substrate feeding assay in sample I. A peak (open arrow) corresponding to glycyrrhetinic acid as sugar acceptor substrate was detected from (b). In addition, a peak (solid arrow) conceivably derived from glycyrrhetinic acid, to which a single glucuronic acid molecule was added, was detected from (c). The retention time and mass spectrum of this peak satisfactorily coincided with those of glycyrrhetinic acid monoglucuronide.

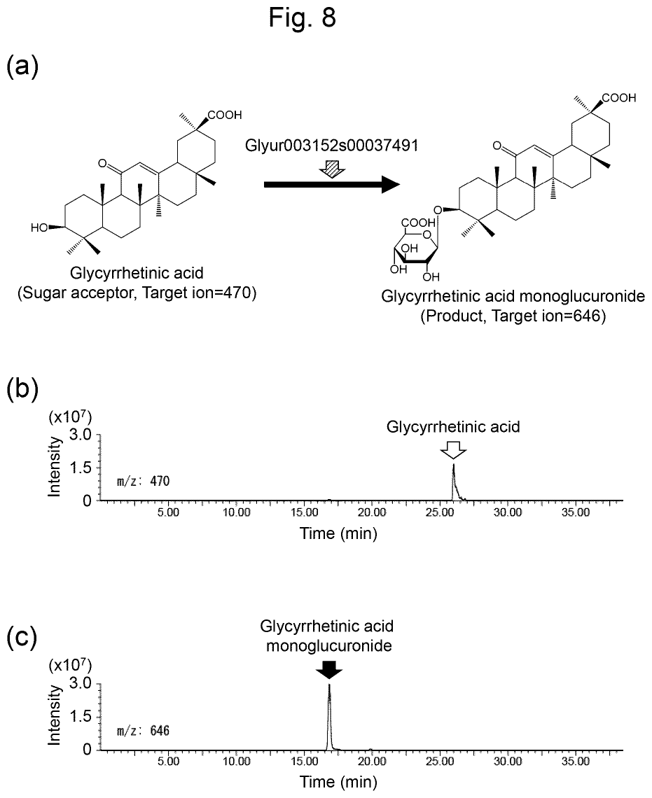

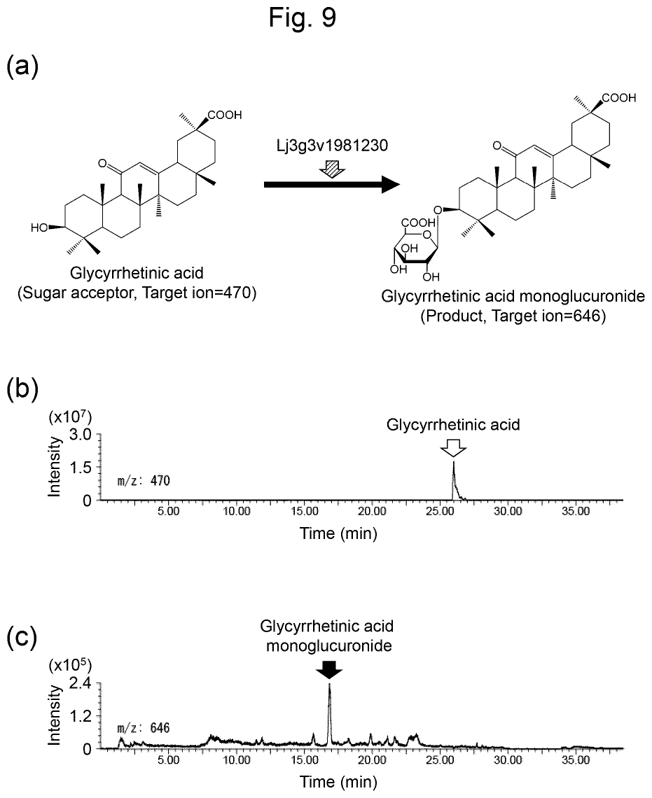

As shown in (b) and (c), respectively, peaks corresponding to glycyrrhetinic acid (open arrow) and glycyrrhetinic acid monoglucuronide (solid arrow) were detected from sample J shown in FIG. 8 and from sample K shown in FIG. 9. The retention time and mass spectrum of each peak satisfactorily coincided with those of glycyrrhetinic acid monoglucuronide.

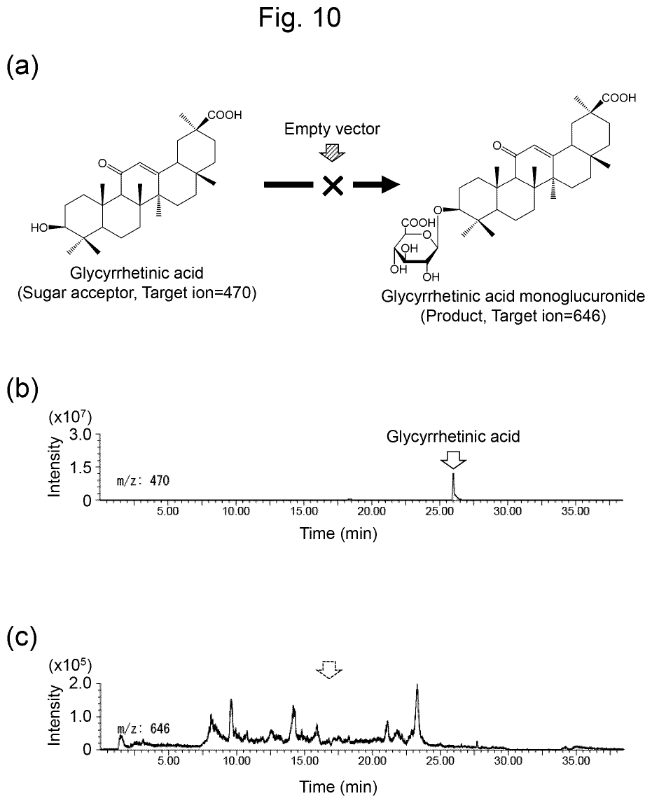

On the other hand, for sample L of a negative control shown in FIG. 10, a peak (open arrow) corresponding to glycyrrhetinic acid as sugar acceptor substrate was detected in (b), but no peak was detected at the position (dash line arrow) corresponding to glycyrrhetinic acid monoglucuronide in (c).

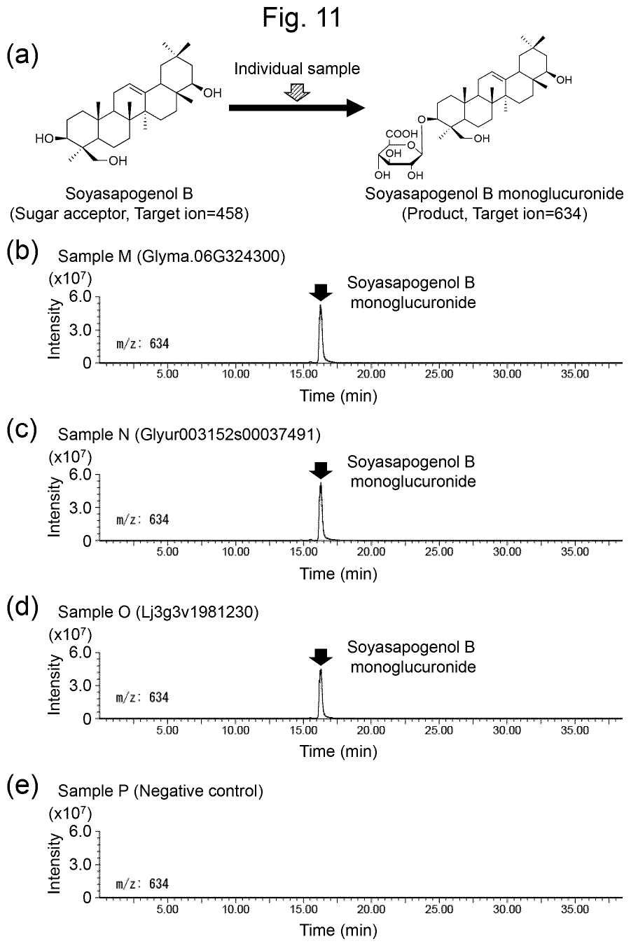

Results of substrate feeding assay using soyasapogenol B as a sugar acceptor substrate are shown in FIG. 11. A peak (solid arrow) corresponding to soyasapogenol B monoglucuronide which is prepared by adding a single glucuronic acid molecule to soyasapogenol B was detected for each of sample M containing soybean-derived Glyma.06G324300 shown in (b), sample N containing Glycyrrhiza-derived Glyur003152s00037491 shown in (c), and sample O containing L. japonicus-derived Lj3g3v1981230 shown in (d). The retention time and mass spectrum of the peak satisfactorily coincided with those of soyasapogenol B monoglucuronide. In contrast, from sample P which is a negative control shown in (e), a peak corresponding to soyasapogenol B monoglucuronide was not detected.

The aforementioned results and results obtained in Example 10 show that the aforementioned novel soybean-derived enzyme Glyma.06G324300 obtained in Example 1, the novel Glycyrrhiza-derived enzyme Glyur003152s00037491 obtained in Example 3, and the novel Lotus japonicus-derived enzyme Lj3g3v1981230 obtained in Example 5 have a glucuronic acid transfer activity 1 which converts glycyrrhetinic acid to glycyrrhetinic acid monoglucuronide by transferring glucuronic acid to the hydroxy group at the 3-position of the glycyrrhetinic acid. It is also demonstrated that they have glucuronic acid transfer activity 1 which converts soyasapogenol B into soyasapogenol B monoglucuronide by transferring glucuronic acid to the hydroxy group at the 3-position of the soyasapogenol B. Accordingly, the novel enzyme thus obtained was identified as glucuronosyltransferase 1 which transfers glucuronic acid to the hydroxy group at the 3-position of an oleanane-type triterpenoid.

### Example 14: Isolation of Lotus japonicus Glyma.06G324300 Homologous Gene Loss-of-Function Mutant

Based on the sequence information of Lotus japonicus gene and protein, and their expression database, Lotus Base (https-colon-slash-slash lotus.au.dk/), mutant lineages having LORE insertion in Lj3g3v1981230 were searched. As a result, 19 lineages were hit. Based on the position of LORE1 insertion in Lj3g3v1981230 and the number of LORE1 insertion in other genes, 2 lineages (30006020, 30115796) were selected from these lineages, and seeds were obtained from a distributor (Aarhus University, Denmark). The seeds were sown, genomic DNA was extracted from some of the unfolded cotyledons, and insertion of LORE1 into Lj3g3v1981230 was confirmed by PCR. A cycle of PCR was repeated 25 times by use of GoTaq (registered trademark) Colorless Master Mix (Promega Corporation) at an annealing temperature of 60 °C and a reaction temperature of 72 °C. For PCR, forward primers (30006020 represented by SEQ ID NO: 26, 30115796 represented by SEQ ID NO: 28) and reverse primers (30006020 represented by SEQ ID NO: 27, 30115796 represented by SEQ ID NO: 29) and P2 primer (SEQ ID NO: 30) were used.

### Example 15: Triterpenoid Saponin Composition Analysis of Glyma.06G324300 Homologous Gene Loss-of-Function Mutant of Lotus japonicus

Whole plants of Glyma.06G324300 homologous gene loss-of-function mutant lineages of Lotus japonicus (30006020, 30115796), after 1 month since they had been seeded in Example 14, were freeze-dried, and then 10 times the dry weight of 80% methanol was added, shaken at room temperature for 1 hour, and centrifuged at 15 krpm for 5 minutes. The supernatant obtained by centrifugation was cleaned using a membrane filter with a pore size of 0.45 μm (GL Chromatodisc 4P, GL SCIENCES), and 2 μL of each extract was subjected to LC-PDA/MS/MS analysis. The apparatuses used were Ultimate 3000SD HPLC/LTQ orbitrap discovery MS (both manufactured by Thermo Fisher Scientific). The extracts were applied to a reverse phase column (C30, Develosil C30-UG-3, Nomura Chemical Co., Ltd.), and saponins were eluted by a linear gradient (20 to 80%/60 min) of acetonitrile containing 0.1% (v/v) formic acid at a flow rate of 0.15 ml/min. The eluate was detected by UV absorption and mass spectrometry (orbitrap-type for parent ion, and ion trap type for fragment ion). The eluate vaporized and positively ionized by the electrospray ionization method was injected in a mass spectrometer. Soyasaponin Bb (m/z=943.52) was used as a sample for analysis, and each saponin molecule was annotated by fragment pattern by MS/MS analysis.

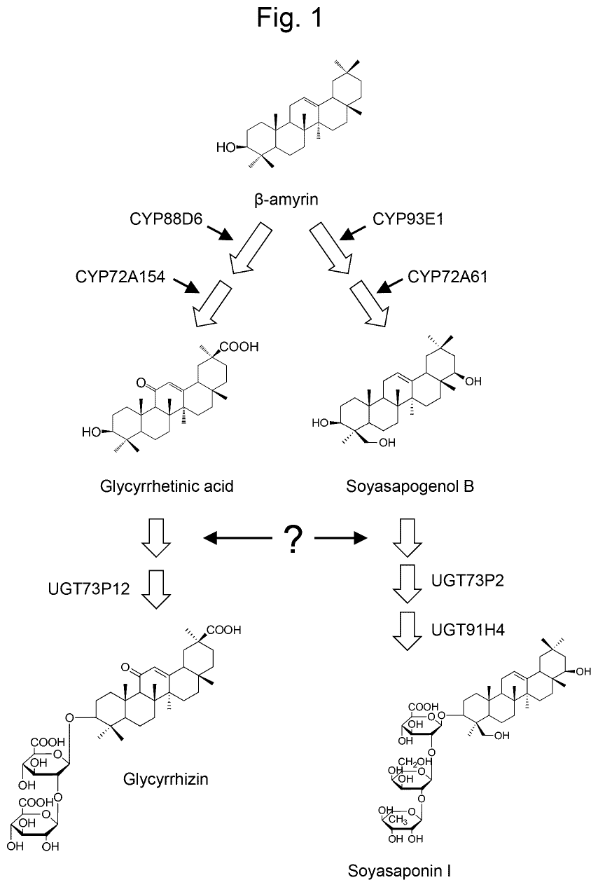

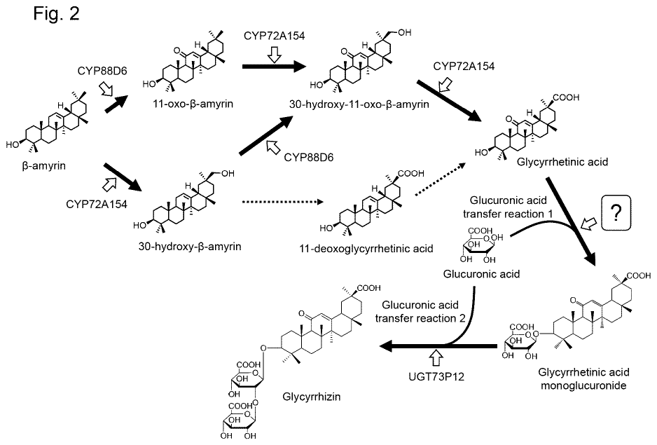

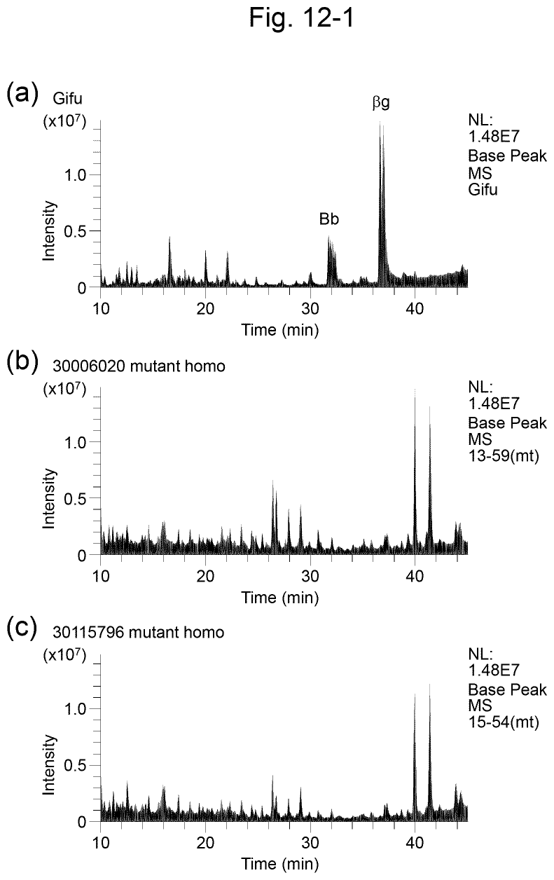

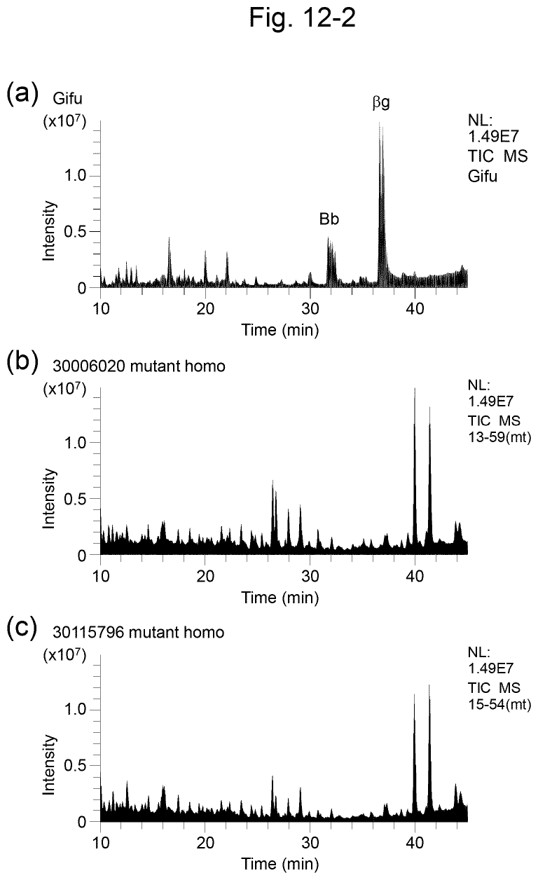

As a result, saponins (Bb, Pg, etc.), normally accumulated in the homozygous mutant (mutant homo), is below a detectable level, and abnormalities were observed in the saponin composition, as shown in (b) and (c) in FIG. 12-1 and FIG. 12-2. The results show that Glyma.06G324300 homologous gene (Lj3g3v1981230) of Lotus japonicus actually functions in vivo in the saponin biosynthetic system.

### Example 16: Construction of Expression Vector for Lotus japonicus

A cycle of PCR was repeated 30 times by use of PrimeSTAR GXL DNA Polymerase (TAKARA BIO INC.) at an annealing temperature of 60 °C and a reaction temperature of 68 °C, using a vector for cloning containing Glycyrrhiza-derived Glyma.06G324300 homologous gene produced in Example 4 as a template and a forward primer (SEQ ID NO: 31) and a reverse primer (SEQ ID NO: 32) that amplify from the initiation codon to the stop codon of SEQ ID NO: 4. The DNA fragment, which was amplified, was cloned to pDONR™221 by the nucleotide sequence specific recombination reaction (GATEWAY attB×attP reaction) using Gateway BP Clonase II Enzyme Mix (Thermo Fisher Technologies). The polynucleotide sequences of the resultant three independent clones were determined, and found to coincide with SEQ ID NO: 4. The plasmid pDONR-Glyur003152s00037491 having the polynucleotide was obtained as an entry clone.

Subsequently, using a vector for cloning containing Lotus japonicus-derived Glyma.06G324300 homologous gene produced in Example 5 as a template, and the forward primer (SEQ ID NO: 33) and the reverse primer (SEQ ID NO: 34) which amplify from the initiation codon to the stop codon of SEQ ID NO: 6, PCR was performed in the same manner as described above to clone into pDONR™221. The polynucleotide sequences of the resultant three independent clones were determined, and found to coincide with SEQ ID NO: 6. The plasmid pDONR-Lj3g3v1981230 having the polynucleotide was obtained as an entry clone. Note that 12 nucleotides represented by SEQ ID NO: 35 (AAAAAGCAGGCT) are artificially added to the 5′ terminal of the forward primer and 12 nucleotides represented by SEQ ID NO: 36 (AGAAAGCTGGGT) are artificially added to the 5′ terminal of the reverse primer, for the reason that the nucleotides are required for nucleotide sequence specific recombination reaction (GATEWAY attB×attP reaction) in cloning into pDONR™221 (Thermo Fisher Technologies). A plasmid (entry clone) pDONR-Glyma.06g324300 having the polynucleotide represented by SEQ ID NO: 2 or a plasmid (entry clone) pDONR-Glyur003152s00037491 having the polynucleotide represented by SEQ ID NO: 4, a plasmid (entry clone) pDONR-Lj3g3v1981230 having the polynucleotide represented by SEQ ID NO: 6, produced in Example 1, and a destination vector pG35NGw were mixed with each other, and subjected to a nucleotide sequence specific recombination reaction (GATEWAY attL×attR reaction) using Gateway LR Clonase II Enzyme Mix (Thermo Fisher Technologies) to transfer the DNA fragment represented by SEQ ID NO: 6 to pCAMBIA-G35NGw. In this manner, a vector pG35N-LjCSL for Lotus japonicus transformation containing Lotus japonicus-derived Glyma.06G324300 homologous gene represented by SEQ ID NO: 6 was obtained. In the same manner as described above, soybean-derived Glyma.06G324300 gene represented by SEQ ID NO: 2, and Glycyrrhiza-derived Glyma.06G324300 homologous gene represented by SEQ ID NO: 4 were transferred to obtain vectors for transformation of Lotus japonicus pG35N-GmCSL and pG35N-GuCSL, respectively.

### Example 17: Rescue Experiment by Introduction of Soybean Glyma.06G324300, and Glycyrrhiza and Lotus japonicus Glyma.06G324300 Homologous Genes into Lotus japonicus-Mutant

Glyma.06G324300 homologous genes were introduced into Lotus japonicus Glyma.06G324300 homologous gene loss-of-function mutant according to the method described in Diaz et al., (2005) Induction of hairy roots for symbiotic gene expression studies. In Lotus japonicus Handbook, A. J. Marquez, ed (Dordrecht, The Netherlands: Springer), pp. 261-277. Seeds obtained from a mutant homozygous lineage of Lotus japonicus Glyma.06G324300 homologous gene loss-of-function mutant 30006020 obtained in Example 14 were sterilized with hypochlorous acid of effective chlorine concentration of 2% (containing 0.02% Tween20) for 20 minutes, then allowed to absorb water in sterile distilled water overnight. The water absorbed seeds were hulled, seeded on 0.8% water agar medium, shaded with aluminum foil, cultured at 25 °C for 4 days, and then illuminated for 1 day. The vector produced in Example 16 was introduced into Agrobacterium (LBA1334), plated on the front surface of L medium, and cultured at 28 °C for 1 day. After Agrobacterium cultured for 1 day was suspended in 10 mL of sterile water and placed in a round sterile petri dish, the seedlings of the mutant homozygous lineage of Lotus japonicus Glyma.06G324300 homologous gene loss-of-function mutant 30006020 were immersed therein and the hypocotyl was cut with a razor. The cut seedlings were placed on a cocultivation medium, shaded with aluminum foil, and co-cultured at 21 °C for 4 days. After co-cultivation, plants were placed on HRE medium and grown for 2 weeks with 16 hours of light period at 25 °C/8 hours of dark period at 23 °C. In the plants that developed hairy roots, GFP fluorescence was confirmed under a fluorescent stereomicroscope.

### Example 18: Triterpenoid Saponin Composition Analysis of Lotus japonicus Hairy Root

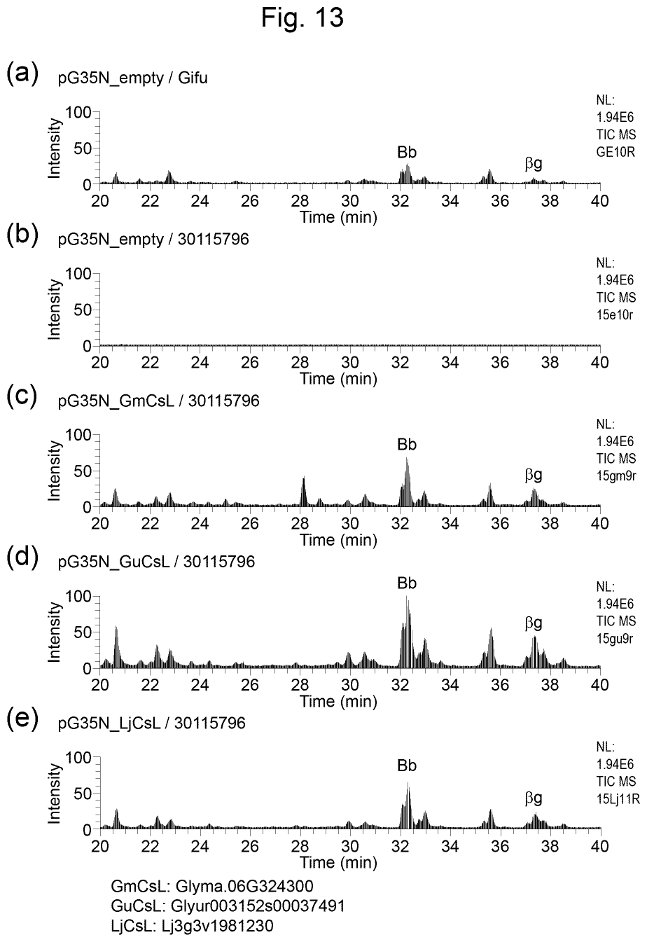

The plants that developed hairy roots obtained in Example 17 were transplanted into pots filled with vermiculite, and B & D hydroponic solution (Diaz et al., 2005) was added and grown for one month. Sufficiently grown plants were freeze-dried and ground in Multi-beads shocker (Yasui Instrument Co., Ltd.) at 2500 rpm for 30 seconds. 80% Methanol, 100 times the weight of the freeze-dried material, was added, and shaken at room temperature for 1 hour, and then centrifuged at 15 krpm for 5 minutes to collect the supernatant. The supernatant was analyzed by LC-PDA/MS/MS using the method shown in Example 15. As a result, the saponins that had disappeared in the mutant were restored in the transformed hairy roots as shown in FIG. 13. Therefore, it was considered that Glyma.06G324300 homologous gene also catalyzes the saponin synthesis reaction in vivo.

### Example 19: Search and Isolation of Astragalus-Derived Glyma.06G324300 Homologous Gene

Glyma.06G324300 homologous gene was searched as a candidate for an orthologue gene of Glyma.06G324300 by gene homology search from Astragalus (Astragalus sinicus) which belongs to the same Fabaceae plant as soybean. A kind of nucleotide sequence, AsCSyGT, which encodes protein exhibiting high amino acid identity with Glyma.06G324300 was found from a sequence database integrating RNA sequence data obtained from roots, stems, and leaves of Astragalus. Using the total RNA (1 μg) obtained, a first-strand cDNA was synthesized by use of SMART RACE cDNA amplification kit (Clontech) in accordance with the attached protocol. A cycle of PCR was repeated 30 times by use of PrimeSTAR Max DNA Polymerase (TAKARA BIO INC.) at an annealing temperature of 55 °C and a reaction temperature of 72 °C, using the first-strand cDNA (2 μL) as a template and oligo DNA molecules corresponding to the N terminal and C terminal of the polypeptide estimated from AsCSyGT, respectively, as a forward primer (SEQ ID NO: 39) and a reverse primer (SEQ ID NO: 40). Note that 4 nucleotides (cacc) are artificially added to the 5′ terminal of the forward primer, for the reason that the nucleotides are required for cloning into pENTR™/D-TOPO (registered trademark) entry vector (Thermo Fisher Technologies). The DNA fragment, which was amplified, was cloned to pENTR™/D-TOPO entry vector. The polynucleotide sequences of the resultant two independent clones were determined. As a result, the nucleotide sequence of Glyma.06G324300 homologous gene of Astragalus thus obtained is represented by SEQ ID NO: 41. The polypeptide sequence estimated from the nucleotide sequence is represented by SEQ ID NO: 42. The amino acid sequence represented by SEQ ID NO: 42 had 77% identity with the amino acid sequence represented by SEQ ID NO: 1.

### Example 20: Isolation of Soybean-Derived Glyma.06G324300 Homologous Gene

Glyma.06G324300 homologous gene was searched as a candidate for a paralogue gene of Glyma.06G324300 by gene homology search from soybean. Using BLAST homology search function in genome information database of soybean, i.e., Soybase (https-colon-slash-slash soybase.org), two types of nucleotide sequences, Glyma.04g255400 and Glyma.11g151800, which may encode proteins having high amino acid identity with Glyma.06G324300, were found. Two types of Glyma.06G324300 homologous gene, Glyma.04g255400 and Glyma.11g151800 were amplified by the method described in Example 1, and cloned into pDONR™221 (Thermo Fisher Technologies). Oligo DNA molecules corresponding to the N terminal and C terminal of the polypeptide estimated from Glyma.04g255400 were respectively used as a forward primer (SEQ ID NO: 43) and a reverse primer (SEQ ID NO: 44), and oligo DNA molecules corresponding to the N terminal and C terminal of the polypeptide estimated from Glyma.11g151800 were respectively used as a forward primer (SEQ ID NO: 45) and a reverse primer (SEQ ID NO: 46). Note that 12 nucleotides (AAAAAGCAGGCT) are artificially added to the 5′ terminal of the forward primer and 12 nucleotides (AGAAAGCTGGGT) are artificially added to the 5′ terminal of the reverse primer, for the reason that the nucleotides are required for nucleotide sequence specific recombination reaction (GATEWAY attB×attP reaction) in cloning into pDONR™221 (Thermo Fisher Technologies). The DNA fragment, which was amplified from seed-derived first-strand cDNA, was cloned to pDONR™221 by the nucleotide sequence specific recombination reaction (GATEWAY attB×attP reaction) using Gateway BP Clonase II Enzyme Mix (Thermo Fisher Technologies). The polynucleotide sequences of the resultant three independent clones were determined. As a result, the sequences thus obtained are represented by SEQ ID NO: 47 and SEQ ID NO: 49. The polypeptide sequences estimated from the sequences are represented by SEQ ID NO: 48 and SEQ ID NO: 50. The amino acid sequences represented by SEQ ID NO: 48 and SEQ ID NO: 50 had identities of 93.9% and 71.1%, respectively, with the amino acid sequence represented by SEQ ID NO: 1.

### Example 21: Introduction of Glyma.06G324300 Homologous Gene of Astragalus and Soybean into Glycyrrhetinic Acid and Soyasapogenol B-Producing Yeast Strain

According to the method described in Example 7, pESC-HIS-AsCSyGT, pESC-HIS-AtUGD2-Glyma04g255400, pESC-HIS-AtUGD2-Glyma. 11g151800, which are yeast expression clones of Glyma.06G324300 homologous genes of Astragalus obtained in Example 19 and soybean obtained in Example 20, were constructed, and introduced into glycyrrhetinic acid and soyasapogenol B-producing yeast strain, respectively, according to the method shown in Example 8.

### Example 22: In Vivo Enzyme Assay Using Recombinant Yeast into which Glyma.06G324300 Homologous Genes of Astragalus and Soybean were Introduced

According to the method described in Example 9, recombinant yeast was cultured, and metabolites were extracted. As a result, a metabolite extract (sample Q) derived from glycyrrhetinic acid-producing yeast strain expressing a polypeptide represented by SEQ ID NO: 42 (AsCSyGT), a metabolite extract (sample R) derived from glycyrrhetinic acid-producing yeast strain expressing a polypeptide represented by SEQ ID NO: 48 (Glyma04g255400), a metabolite extract (sample S) derived from glycyrrhetinic acid-producing yeast strain expressing a polypeptide represented by SEQ ID NO: 50 (Glyma.11g151800) were obtained. In the same manner, soyasapogenol B-producing yeast strains having pESC-HIS-AsCSyGT, pESC-HIS-AtUGD2-Glyma04g255400, pESC-HIS-AtUGD2-Glyma.11g151800 were also cultured, and the metabolites were extracted. As a result, a metabolite extract (sample T) derived from soyasapogenol B-producing yeast strain expressing a polypeptide represented by SEQ ID NO: 42 (AsCSyGT), a metabolite extract (sample U) derived from soyasapogenol B-producing yeast strain expressing a polypeptide represented by SEQ ID NO: 48 (Glyma04g255400), and a metabolite extract (sample V) derived from soyasapogenol B-producing yeast strain expressing a polypeptide represented by SEQ ID NO: 50 (Glyma.11g151800) were obtained.

### Example 23: Analysis of Metabolite Extract of Yeast into which Glyma.06G324300 Homologous Genes of Astragalus and Soybean were Introduced

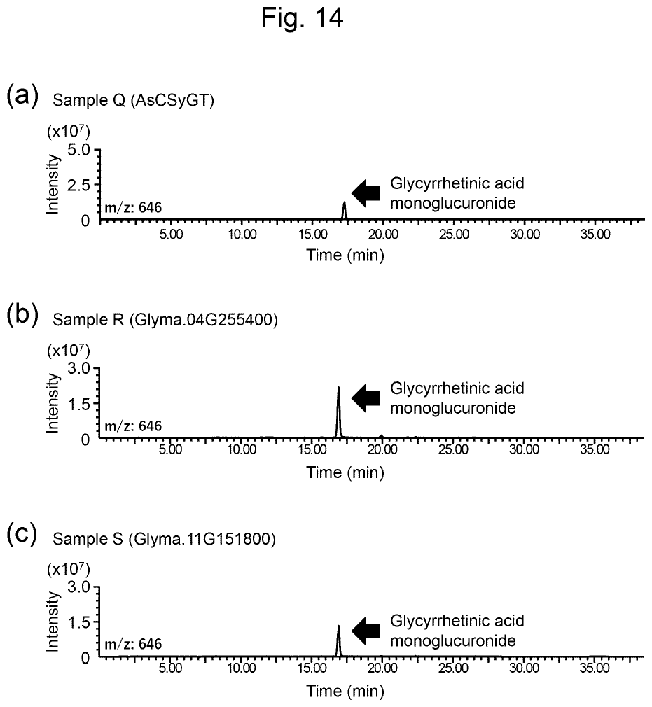

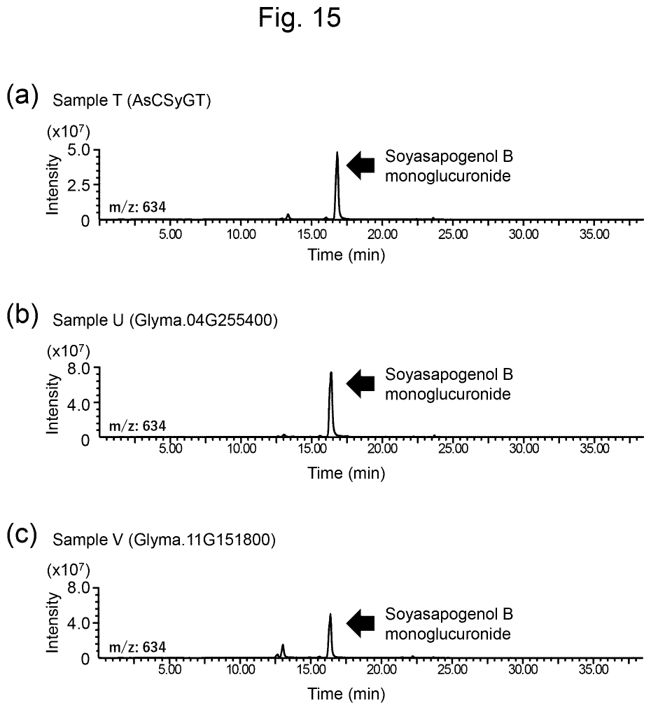

According to the method described in Example 10, samples for LC-MS analysis were prepared, and analyzed. The results are shown in FIG. 14 and FIG. 15.

A peak (solid arrow) corresponding to glycyrrhetinic acid monoglucuronide was detected in sample Q in (a) shown in FIG. 14. The retention time and mass spectrum of this peak satisfactorily coincided with those of glycyrrhetinic acid monoglucuronide. Similarly, a peak (solid arrow) corresponding to glycyrrhetinic acid monoglucuronide was detected in sample R in (b) and sample S in (c). The retention time and mass spectrum of each peak satisfactorily coincided with those of glycyrrhetinic acid monoglucuronide.

A peak (solid arrow) corresponding to soyasapogenol B monoglucuronide was detected in sample T in (a) shown in FIG. 15. A peak (solid arrow) corresponding to soyasapogenol B monoglucuronide was also detected in sample U in (b) and sample V in (c). The retention time and mass spectrum of each peak satisfactorily coincided with those of soyasapogenol B monoglucuronide.

The aforementioned results show that Astragalus-derived AsCSyGT obtained in Example 19, and soybean-derived Glyma04g255400 and Glyma.11g151800 obtained in Example 20 have a glucuronic acid transfer activity 1 which converts glycyrrhetinic acid into glycyrrhetinic acid monoglucuronide by transferring glucuronic acid to the hydroxy group at the 3-position of the glycyrrhetinic acid. It is also demonstrated that they have glucuronic acid transfer activity 1 which converts soyasapogenol B into soyasapogenol B monoglucuronide by transferring glucuronic acid to the hydroxy group at the 3-position of the soyasapogenol B.

### Example 24: Preparation of Transformed Yeast for Substrate Feeding Assay of Glyma.06G324300 Homologous Gene of Soybean

pESC-HIS-AtUGD2-Glyma04g255400 and pESC-HIS-AtUGD2-Glyma.11g151800 obtained in Example 21, were respectively introduced into the yeast INVScI strains. The yeast was transformed by use of Frozen-EZ Yeast Transformation II (Zymo Research) in accordance with the attached protocol.

### Example 25: Substrate Feeding Assay Using Transformed Yeast into which Glyma.06G324300 Homologous Gene of Soybean was Introduced

The recombinant yeast obtained in Example 24 was cultured by the method shown in Example 12. The resulting suspension of each yeast cell was aliquoted and ursolic acid of ursane-type triterpenoid or betulinic acid of lupin-type triterpenoid at a final concentration of 5 μM was added, respectively. Thereafter, culture and extraction of metabolite were performed again using the method shown in Example 12. As a result, feeding assay extracts including a transformed yeast expressing polypeptide represented by SEQ ID NO: 48 (Glyma04g255400) or polypeptide represented by SEQ ID NO: 50 (Glyma.11g151800), with an ursane-type triterpenoid, i.e., ursolic acid, added thereto, and those with lupane-type triterpenoid, i.e., betulinic acid, added thereto, were obtained.

### Example 26: Analysis of Substrate Feeding Assay Extract Using Transformed Yeast into which Glyma.06G324300 Homologous Gene of Soybean was Introduced

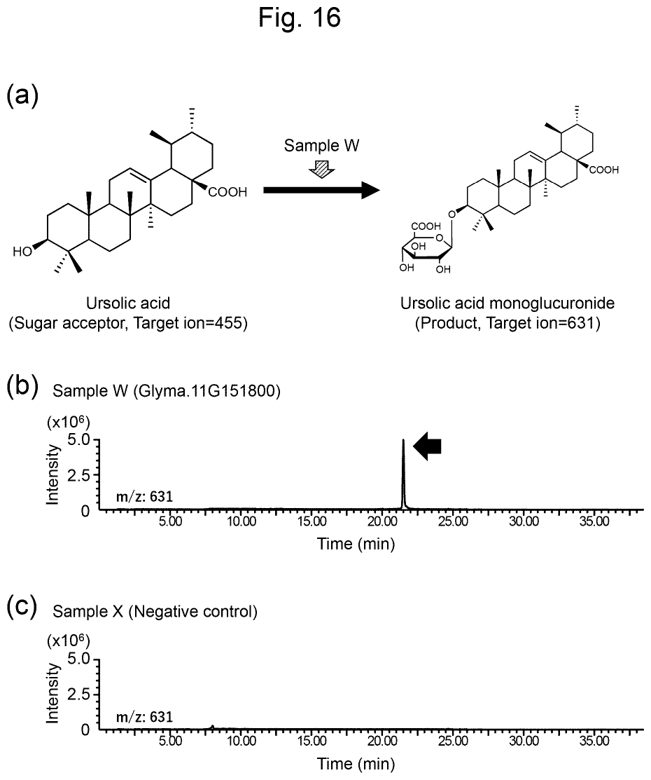

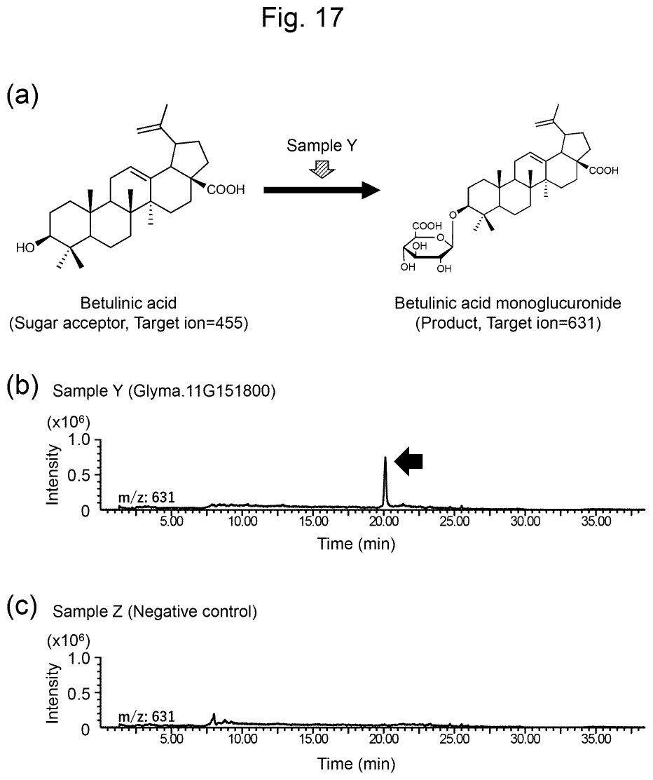

Samples obtained in Example 25 were analyzed in the same manner as in Example 10, using SIM mode with the following parameters in MS: m/z of each expected reaction product, ursolic acid monoglucuronide=631 (FIG. 16, a), betulinic acid monoglucuronide=631 (FIG. 17, a). FIG. 16 shows analysis results of a feeding assay extract (sample W) including a transformed yeast expressing a polypeptide represented by SEQ ID NO: 50 (Glyma.11g151800) to which ursolic acid was added. A peak estimated as ursolic acid monoglucuronide was detected in sample W shown in (b). In contrast, in sample X which is a negative control shown in (c), a peak estimated as ursolic acid monoglucuronide was not detected.

FIG. 17 shows analysis results of a feeding assay extract (sample Y) including a transformed yeast expressing a polypeptide represented by SEQ ID NO: 50 (Glyma.11g151800) to which betulinic acid was added. A peak estimated as betulinic acid monoglucuronide was detected in sample Y shown in (b). In contrast, from sample Z which is a negative control shown in (c), a peak estimated as betulinic acid monoglucuronide was not detected.

Based on the above, it is considered that the polypeptide (Glyma.11g151800) represented by SEQ ID NO: 50 is glucuronosyltransferase 1 capable of transferring glucuronic acid to the hydroxy group at the 3-position not only of oleanane-type triterpenoid, but also of ursane-type triterpenoid such as ursolic acid and B-boswellic acid, and of lupane-type triterpenoid such as betulinic acid.

All publications, patents and patent applications cited in the specification as references are incorporated in their entirety herein.

1-3. (canceled)

4. A recombinant cell for producing glycyrrhetinic acid monoglucuronide, wherein the recombinant cell is obtained from a non-animal, capable of biologically synthesizing β-amyrin, and comprises all of the following polynucleotides (1) to (3):
(1) a polynucleotide encoding a polypeptide having an activity of oxidizing the 11-position in an oleanane-type triterpenoid and consisting of an amino acid sequence of the following (a) or (b):
(a) the amino acid sequence of SEQ ID NO: 7, or
(b) an amino acid sequence having at least 90% sequence identity with the amino acid sequence of SEQ ID NO: 7;
(2) a polynucleotide encoding a polypeptide having an activity of oxidizing the 30-position in an oleanane-type triterpenoid and consisting of an amino acid sequence of the following (c) or (d):
(c) the amino acid sequence of SEQ ID NOs: 9, 11, or 13, or
(d) an amino acid sequence having at least 90% sequence identity with the amino acid sequence shown of SEQ ID NOs: 9, 11, or 13,
(3) a polynucleotide encoding a polypeptide having an activity to transfer glucuronic acid to the hydroxy group at the 3-position in an oleanane-type triterpenoid, and consisting of an amino acid sequence of the following (e) or (f):
(e) the amino acid sequence of SEQ ID NO:3 or 5, or
(f) an amino acid sequence polypeptide having at least 90% sequence identity with the amino acid sequence of SEQ ID NO: 3 or 5.

5. The recombinant cell according to claim 4, wherein the cell is a yeast cell.

6. A method for producing glycyrrhetinic acid monoglucuronide from β-amyrin, comprising culturing the recombinant cell of claim 4.

7. A method for producing glycyrrhetinic acid monoglucuronide from glucose, comprising culturing the recombinant cell of claim 4 in a glucose-containing medium.

8. A method for producing glycyrrhetinic acid monoglucuronide from β-amyrin, comprising culturing the recombinant cell of claim 5.

9. A method for producing glycyrrhetinic acid monoglucuronide from glucose, comprising culturing the recombinant cell of claim 5 in a glucose-containing medium.


SEQUENCE LISTING (52 sequences)

SEQ ID NO: 1 - protein, 699 aa, Glycine max
MAMFTYHVETVQSWLALSRLHILIHLVAVLSLCYYRITHLLLEPPTAPWLLMTVAELLLSVLWFFNQAFRWRPVSRSVMTEKLPRDEKLPGLDIFVCTLDPEKEPTVEVMDTIISAVAMDYPSDKLAVYLSDDGGCDVTLYGIREAAEFAKEWVPFCNIYGVKSRCPKVFFSPFGEEDQHTLRHDGFSTQRDLIKAKYEKMQKNIEKFGSDPKNRRIVSDRPPRIEIINDQPGMPLVVYVSRERRPSLPHKFKGGALNALLRVSGLISNGPYVLAVDCDMYSNDPTSAKQAMCFFLDPETSKYIAFVQFPQMFHNLSKKDIYDNQSRTAFKTMWQGMDGLRGPGLSGSGNYLSRSALLFGSPNQKDDYLKDAQKYFGKSTAYIESLKAIRGQKSSKKNISRDEMLREAQVVASCSYENNTNWGTEVGFSYGILLESTITGYLLHSRGWKSAYLYPKTPCFLGCAPTDIKEGMLQLVKWLSELLLLGVSSKYSPFTYGFSRMSIIHTFTYCFMTMSSLYAVVFILYGIVPQVCLLKGITVFPKATDPWFAVFAFVYVSTQIQHLIEVLSGDGSVAMWWDEQRIWILKSVTSIFAIIDGIKKWLGLNKVKFNLSNKAIDKEKLKKYEQGRFDFQGAAVFMAPLVLLLIANIVSFFVGIWRLFNFNVKDFEEMFGQLFLVTYVMLLSYPILEAIVTMKSKSG

SEQ ID NO: 2 - DNA, 2100 bp, Glycine max
  source 1..2100: mol_type = other DNA
atggcaatgttcacgtaccacgttgaaacggttcaatcatggttggcactgagcagacttcacatactgatccacttagtggcagtgttgtcgctgtgttactaccgcataactcacttgttactggaacctccaacagcgccatggcttctgatgaccgtagcggagcttcttctctcggtgctctggttcttcaaccaagccttccggtggcggccggtgtcgcggagcgtcatgacggagaagctgccgagggatgagaagctgccggggcttgacatcttcgtgtgcacgctggatcccgagaaggagcccaccgtggaggtcatggacaccatcatctccgcggtggccatggattacccctccgacaagcttgccgtgtatctctccgacgacggcggctgtgacgttactctctatgggatcagagaggctgctgagttcgccaaggagtgggttccgttctgtaacatttatggggtcaagtcaaggtgtcccaaggtcttcttctctccctttggggaggaggatcaacacactcttcgccacgatggattcagtacacaacgagacctcatcaaggctaaatacgagaagatgcagaaaaatatcgagaaatttggctcagaccctaaaaatcgtcgtattgtgagtgacagacctcctcgtattgagattataaatgaccaaccgggaatgccacttgttgtttatgtgtctcgcgaaagaaggccatcccttcctcacaaattcaaaggaggagccctcaacgcattgctcagagtctcaggtttaatcagtaatggaccttatgttctagcagtggattgtgatatgtatagcaatgatccaacctctgccaaacaagccatgtgcttctttcttgatcctgaaacgtccaaatatattgcatttgttcaattccctcaaatgtttcacaaccttagcaaaaaagacatctatgataatcaatctagaactgcttttaagactatgtggcaaggcatggatggactaagaggtccaggtctttcaggcagcggcaattacttaagtagaagtgctttactatttggaagtccaaaccaaaaagacgactatctgaaggatgcacaaaagtactttggcaagtctaccgcatacattgaatcactgaaggccatccgtggacagaaaagtagcaaaaagaatatttcaagagatgaaatgttaagagaagctcaagtagtggcctcttgttcctacgaaaataacacaaattggggcacagaggtgggattctcatatggcatattactagagagtactattactggctatcttcttcacagcagaggatggaaatcagcatatctttaccccaaaacaccatgtttcttaggttgtgctcccactgacatcaaggaaggcatgcttcagttggttaagtggttgtctgaacttttgttgcttggtgtctcctccaaatacagcccattcacttatggattttcaagaatgtccataattcacaccttcacttattgcttcatgacaatgtcatccctttatgctgttgtcttcatcctctatggcattgtacctcaagtgtgcctcctcaaaggaatcactgtgtttccaaaggccacggacccttggtttgcagtgtttgcatttgtgtatgtatctactcaaattcaacatttgattgaggttctatctggggatggctctgtggctatgtggtgggatgaacaaagaatttggattctgaagtcagttaccagcatatttgcaattatagatggaatcaagaagtggctggggttgaacaaggtgaaattcaacttgtcaaacaaagccattgacaaggagaagctcaagaaatatgagcaaggtaggttcgatttccaaggtgcagctgtgttcatggctccattggtcctattactcatagccaacattgttagcttctttgttggtatatggagactattcaatttcaatgtgaaggattttgaagaaatgtttggtcaacttttcctagtcacctatgtaatgcttcttagttatcccattcttgaggccatagtaacaatgaaaagcaaaagtggatag

SEQ ID NO: 3 - protein, 695 aa, Glycyrrhiza uralensis
MASFTLHTETVQSWLLLSRLHILLHLAVVLLLLYYRITRFPFHAPTLPWTLMTVGEAIMAVLWFFNQAFRWRPVSRSVMTEKLPSDAKLPGLDIFVCTLDPEKEPTVEVMNTLVSALAMDYPPDKLSVYLSDDGAAPVTLYGVREASEFARVWVPFCKKYGIKSRCPKVFFSPSAEDEHLLRTDEFRSERDLIKAKYEKMQKNIEKFGSDAKNCRMVTDRPPRIEILIDQPDMPRVVYVSRERRPSLPHKFKGGALNTLLRVSGLISNGPYVLVVDCDMYCNDPSSAKQAMCFFLDPETSKYIAFVQFPQMFHNLGKKDIYDNQSRTAFKTMWQGMDGLRGPGLSGSGNYLNRSALLFGSPNQKDDYLDDAQNYFGKSTMYIESLEAIRGQKTMKKNISRDEILREAQVLASCSYETNTEWGAEVGFSYGILLESSITGYLLHCRGWKSAYLYPKTPCFLGCAPTDIKEGMLQLVKWLSEYCLLGFSKYSPFTYGFSRMPIMPTLVYCFLTTTTLYSIVFILYGIVPQVCFLKGIPVFPKVTDPWFAVFATLYISTQIQHLIEVLSGDGSVAMWWDEQRIWILKSVTSVFAIIEAAKKGLGLNKKKFMLSNKAIDKEKLKKYEQGRFDFQGAALFMSPMVVLLIVNVVSFIGGIWRLFNAKDIEDMFGQLFLVSYVMALSYPIFEGIITMKSKSG

SEQ ID NO: 4 - DNA, 2088 bp, Glycyrrhiza uralensis
  source 1..2088: mol_type = other DNA
atggcaagcttcacccttcacacagaaaccgttcagtcatggctactcctcagcagacttcacatactgctgcacctcgcagttgtactgctcctcttatactaccgcatcacacgtttccccttccatgctccgactctaccgtggactctgatgaccgtaggtgaggctattatggcagtgctgtggttcttcaaccaggccttccggtggcggccggtgagccgctcggtgatgacggagaagctgcccagcgacgcgaagctgccggggcttgacatattcgtgtgcacgcttgaccccgagaaggagcccaccgtggaggtgatgaacactctggtctctgcccttgccatggactacccccctgacaagctctccgtttacctctccgacgatggcgccgccccggtcactctttacggcgtgagagaggcttctgagttcgcgagggtgtgggtccctttctgcaaaaagtatgggatcaagtcaaggtgtcccaaggttttcttctctcccagtgctgaggatgaacaccttcttcgcaccgacgagttcaggtcagagcgagacctcatcaaggctaaatacgagaaaatgcagaaaaatatcgagaaatttggttcggatgccaaaaattgtcgtatggtgaccgacagacctcctcggatcgagatattgattgaccaaccagacatgccacgtgttgtttacgtgtctcgggaaagaaggccatcactccctcacaagttcaaaggaggagccctcaatacattgctcagagtctcaggtctaatcagcaatgggccttatgtacttgtagtggactgtgatatgtattgcaatgacccatcctcagccaaacaagccatgtgtttctttcttgatcctgaaacctctaaatatattgcatttgtccaattccctcaaatgtttcacaaccttggcaaaaaagacatctatgacaatcaatctaggactgcttttaagacaatgtggcaagggatggatggactaagaggtcctggtctttctggcagcggtaattacttgaatagaagtgcattactatttggaagtccaaatcaaaaagatgactatctggatgatgcccaaaactacttcggcaagtctaccatgtacatagaatcactagaggccattcgtggacaaaaaactatgaaaaagaatatttcaagagatgaaattttacgagaagctcaagtattagcctcttgttcctatgagacaaacacagaatggggcgcagaggtaggattctcatatggcatcttactggagagttcaatcactggctatcttttgcactgcagaggatggaaatcagcatatctttacccaaagacaccatgtttcttagggtgtgccccaactgacatcaaggaaggaatgctccaattggtgaagtggttgtctgaatactgcttgcttggattctctaaatacagccctttcacttatggcttttcaagaatgcccattatgcctaccttagtctattgcttcttgacaactacaaccctttattccattgtcttcatcctttatggcattgtcccccaagtttgcttcttaaaaggaatacccgtgtttccaaaggtcacagacccttggtttgcagtgtttgcaacactgtatatatccacccagattcaacatttgatagaggtcctttctggtgatggctctgtggcaatgtggtgggatgaacagcgaatctggattctgaagtcagtcactagcgtgttcgcaatcatagaggcagctaagaaagggttaggattgaacaagaagaaattcatgttgtcaaacaaagcaattgacaaggagaagctcaagaagtatgagcaaggtaggtttgatttccaaggtgcagctctgttcatgtccccaatggttgtgttgctcatagtgaacgttgtttccttcattggtggcatatggagactattcaatgcaaaggatattgaagatatgtttggtcagcttttcctagttagttatgtaatggcccttagttatcccatttttgaagggataataaccatgaaaagcaagagtggataa

SEQ ID NO: 5 - protein, 694 aa, Lotus japonicus
MANFTLHTETVQAWLPLSRLHILIHSVFVILLLYYRTTRLIHAPTAPWILMTVAEALLAVLWLFNQAFRWRPVSRSVKTEKLPRDENLPGLDIFVCTIDPEKEPTAGVMDTVVSAVAMDYPPDKLSVYLSDDGGCAVTEYGIREACEFAKVWVPFCRKYGIKSRCPKVFFSPMGEDEEILRTDEFRAEQEKIKAQYETMQKNIEKFGSDPKNCRIVTDRPSKIEIINEQSEIPRVVYVSRERRPSLPHKFKGGALNTLLRVSGLISNGPYVLAVDCDMYCNDPSSAKQAMCFFLDPETSKYIAFVQFPQMFHNLSKKDIYDNQSRTAFKTMWQGMDGLSGPGLSGSGNYLSRSALLFGSPNQKDDYLLDAQNYFGESPLYIESLKAIRGQQTTKKNISRDESLLEAKVVASASYETNTEWGSEVGFSYGILLESTITGYLLHCRGWKSAYLYPKTPCFLGCAPTDIKEGMLQLVKWLSELCLFAVSKYSPFTYGFSRLPIMPTFTYCFLAASSLYAIVFILYGIVPQVCFLKGIPVFPKATDPWFAVFAVLYVATQIQHLIEVLSGNGSVSMWWDEQRIWILKSVTSVFAMIEGIKKWLGLNKKKFNLSNKAVDKEKVKKYEQGRFDFQGAALYMSPMVVLLLVNIVCFFGGLWRLFKEKDFADMFGQLFLLSYVMALSYPILEGIVTMKMKSG

SEQ ID NO: 6 - DNA, 2085 bp, Lotus japonicus
  source 1..2085: mol_type = other DNA
atggccaatttcactctccacacagaaaccgttcaagcatggctccctctaagcagactccacattcttatacactcagtgttcgtcatccttctcctctactaccgcacaacgcgtctcatccacgcgccgaccgcgccgtggatcctgatgaccgttgcggaggctctcctcgccgtgctttggctcttcaaccaggccttccggtggcgaccggtgagccgctccgtgaagacagagaagctgccgcgcgacgagaatctccccgggctggacatatttgtgtgcacgattgatcctgagaaggagccaacggcaggggtgatggacacggttgtttccgccgtggcgatggattacccgccggataagctatccgtgtatctttctgatgatggtggttgcgccgtgacggagtatgggattagagaggcttgtgagtttgccaaggtgtgggttcctttttgtagaaagtatgggatcaagtcgaggtgtccaaaagttttcttctctccgatgggggaagatgaagagattctaaggacagatgagttcagagcagagcaagagaagatcaaggcccaatacgagactatgcagaaaaacatcgagaaatttggttcagaccccaaaaattgtcgtattgtcactgacagaccctctaagatcgagattataaatgagcaatcagaaatcccacgtgttgtgtacgtctctcgtgaaagaaggccatcacttcctcacaagttcaaaggaggagctctcaacacattgctcagagtgtcaggtctaatcagcaatggaccttatgtgcttgcagtggattgtgatatgtattgcaatgatccatcctctgccaagcaagcaatgtgcttcttccttgatccagaaacatctaaatacattgcatttgtccaattccctcaaatgtttcacaaccttagtaagaaagacatctatgataatcaatctaggactgcttttaagacaatgtggcaaggcatggatggactcagtggtccaggtctttctggcagtggtaactacttgagtagaagtgcattgctatttggaagtccaaaccaaaaagatgactatctgcttgatgctcaaaactactttggcgagtctcccttgtacattgaatcattgaaggccatccgtggacaacaaactaccaaaaagaatatctcaagagacgaaagtttactagaagctaaagtggtggcctctgcttcctacgagacaaacacagaatggggctcagaggttggattctcatatggcatcttactggagagtactattactggttaccttttgcactgcagaggatggaaatcagcttatctttacccaaaaacaccatgtttcttagggtgtgcccccactgacattaaagaaggcatgcttcagttggtgaagtggttgtctgagctttgcttgtttgctgtctctaagtacagcccttttacatatgggttttcaagattgcccattatgcctaccttcacttattgtttcctggcagcttcatccctatatgctattgtcttcatcctttatggcattgtacctcaagtgtgcttcttgaaaggaatccctgtgtttccaaaggccacagacccttggtttgcagtgtttgcagtattgtatgtagccacccagattcaacatttgattgaagtcctttctggcaatggctcggtctcgatgtggtgggatgaacaaagaatttggattctgaagtcagttactagcgtatttgcaatgatagagggaatcaagaaatggttaggattgaacaagaaaaaattcaacctgtcaaacaaagcggttgacaaggagaaggtcaagaaatatgagcaaggtaggtttgatttccaaggagcagctctgtacatgtctccaatggttgtgttgctcctagtgaacattgtttgcttctttggcggtttatggagactgtttaaggagaaagattttgcagatatgtttggtcaacttttcctactcagctatgtgatggctctcagttatcccattcttgaggggatagtaactatgaaaatgaagagtgggtag

SEQ ID NO: 7 - protein, 493 aa, Glycyrrhiza uralensis
MEVHWVCMSAATLLVCYIFGSKFVRNLNGWYYDVKLRRKEHPLPPGDMGWPLIGDLLSFIKDFSSGHPDSFINNLVLKYGRSGIYKTHLFGNPSIIVCEPQMCRRVLTDDVNFKLGYPKSIKELARCRPMIDVSNAEHRLFRRLITSPIVGHKALAMYLERLEEIVINSLEELSSMKHPVELLKEMKKVSFKAIVHVFMGSSNQDIIKKIGSSFTDLYNGMFSIPINVPGFTFHKALEARKKLAKIVQPVVDERRLMIENGPQEGSQRKDLIDILLEVKDENGRKLEDEDISDLLIGLLFAGHESTATSLMWSITYLTQHPHILKKAKEEQEEITRTRFSSQKQLSLKEIKQMVYLSQVIDETLRCANIAFATFREATADVNINGYIIPKGWRVLIWARAIHMDSEYYPNPEEFNPSRWDDYNAKAGTFLPFGAGSRLCPGADLAKLEISIFLHYFLRNYRLERINPECHVTSLPVSKPTDNCLAKVIKVSCA

SEQ ID NO: 8 - DNA, 1482 bp, Glycyrrhiza uralensis
  source 1..1482: mol_type = other DNA
atggaagtacattgggtttgcatgtccgctgccactttgttggtatgctacatttttggaagcaagtttgtgaggaatttgaatgggtggtattatgatgtaaaactaagaaggaaagaacacccactacccccaggtgacatgggatggcctcttatcggcgatctattgtccttcatcaaagatttctcatcgggtcaccctgattcattcatcaacaaccttgttctcaaatatggacgaagtggtatctacaagactcacttgtttgggaatccaagcatcattgtttgtgagcctcagatgtgtaggcgagttctcactgatgatgtgaactttaagcttggttatccaaaatctatcaaagagttggcacgatgtagacccatgattgatgtctctaatgcggaacataggctttttcgacgcctcattacttccccaatcgtgggtcacaaggcgctagcaatgtacctagagcgtcttgaggaaattgtgatcaattcgttggaagaattgtccagcatgaagcaccccgttgagctcttgaaagagatgaagaaggtttcctttaaagccattgtccacgtcttcatgggctcttccaatcaggacatcattaaaaaaattggaagttcgtttactgatttgtacaatggcatgttctctatccccattaacgtacctggttttacattccacaaagcactcgaggcacgtaagaagctagccaaaatagttcaacccgttgtggatgaaaggcggttgatgatagaaaatggtccacaagaagggagccaaagaaaagatcttattgatattcttttggaagtcaaagatgagaatggacgaaaattggaggacgaggatattagcgatttattaatagggcttttgttcgctggccatgaaagtacagcaaccagtttaatgtggtcaattacgtatcttacacagcatccccatatcttgaaaaaggctaaggaagagcaggaagaaataacgaggacaagattttcctcgcagaaacaattaagtcttaaggaaattaagcaaatggtttatctttctcaggtaattgatgaaactttacgatgtgccaatattgcctttgcaacttttcgagaggcaactgctgatgtgaacatcaatggttatatcataccaaagggatggagagtgctaatttgggcaagagccattcatatggattctgaatattacccaaatccagaagaatttaatccatcgagatgggatgattacaatgccaaagcaggaaccttccttccttttggagcaggaagtagactttgtcctggagccgacttggcgaaacttgaaatttccatatttcttcattatttcctccgtaattacaggttggagagaataaatccagaatgtcacgttaccagcttaccagtatctaaacccacagacaattgtctcgctaaggtgataaaggtttcatgtgcttag

SEQ ID NO: 9 - protein, 523 aa, Glycyrrhiza uralensis
MDASSTPGAIWVVLTVILAAIPIWVCHMVNTLWLRPKRLERHLRAQGLHGDPYKLSLDNSKQTYMLKLQQEAQSKSIGLSKDDAAPRIFSLAHQTVHKYGKNSFAWEGTAPKVIITDPEQIKEVFNKIQDFPKPKLNPIAKYISIGLVQYEGDKWAKHRKIINPAFHLEKLKGMLPAFSHSCHEMISKWKGLLSSDGTCEVDVWPFLQNLTCDVISRTAFGSSYAEGAKIFELLKRQGYALMTARYARIPLWWLLPSTTKRRMKEIERGIRDSLEGIIRKREKALKSGKSTDDDLLGILLQSNHIENKGDENSKSAGMTTQEVMEECKLFYLAGQETTAALLAWTMVLLGKHPEWQARARQEVLQVFGNQNPNFEGLGRLKIVTMILYEVLRLYPPGIYLTRALRKDLKLGNLLLPAGVQVSVPILLIHHDEGIWGNDAKEFNPERFAEGIAKATKGQVCYFPFGWGPRICVGQNFALLEAKIVLSLLLQNFSFELSPTYAHVPTTVLTLQPKHGAPIILHKL

SEQ ID NO: 10 - DNA, 1572 bp, Glycyrrhiza uralensis
  source 1..1572: mol_type = other DNA
atggatgcatcttccacaccaggggctatctgggttgttctgacagtgatactagctgcgattcccatatgggtatgccatatggtgaacacgctgtggctgaggccaaagaggttggaaaggcatctcagagctcaaggtcttcatggcgacccttacaagctctcacttgacaactccaagcaaacctatatgctcaagttgcaacaagaagcacaatcaaaatccattggtctctccaaagatgatgctgcaccacgaatcttctcccttgcccatcaaactgtacacaaatatggaaagaactcctttgcatgggaagggacagcaccaaaggtgatcatcacagacccagagcaaattaaggaagtctttaacaagattcaggacttccccaaaccaaaattaaatcccatcgccaagtatattagcatcggtctagtacagtatgagggtgacaaatgggccaaacatcgaaagattatcaatccggcattccacttagaaaaattgaaaggtatgctgccagcattttctcatagctgccatgaaatgattagcaaatggaaggggttattgtcatcagatggaacatgtgaggttgatgtttggcccttccttcaaaatctcacttgtgatgtaatttctaggacggcattcggaagcagctatgcagaaggagcaaaaatatttgaacttttgaaaaggcagggatatgctttgatgacagcacgatacgcacgcattccattatggtggcttctaccatcaactaccaaaaggaggatgaaggaaattgaaagaggcatacgtgattcacttgaaggtatcattagaaaacgagaaaaagcattgaagagtggcaaaagcaccgatgacgacttattaggcatacttttgcaatcaaatcacattgaaaataaaggagatgaaaacagtaagagtgctggaatgaccacccaagaagtaatggaggaatgcaaacttttttacctggcagggcaagagaccaccgcggctttgctggcctggacaatggtgttattaggcaagcatcctgaatggcaagcacgtgcaaggcaggaagttttgcaagtttttgggaatcaaaatccaaacttcgaagggttaggtcgcctcaaaattgtaaccatgattttatatgaggtactcaggctgtacccacctgggatttacctcacccgagctcttcgaaaggatttgaaacttggaaaccttttgctacctgctggagtacaggtttccgtaccaatacttttgattcaccatgatgaaggtatatggggcaatgatgcaaaggagttcaatcctgaaaggtttgctgaaggaattgcaaaggcaacaaaaggccaagtttgctatttcccttttggatggggtcctagaatatgtgttgggcaaaactttgccttattagaagccaagattgtattgtcattgctgctgcagaatttctcatttgagctatctccgacttatgcacatgttcctaccacggtgcttactttgcagccaaaacatggggcacccatcattctgcataaactgtaa

SEQ ID NO: 11 - protein, 523 aa, Glycyrrhiza glabra
MDASSTPGAIWVVLTVILAAIPIWACHMVNTLWLRPKRLERHLRAQGLHGDPYKLSLDNSKQIYMLKLQQEAQSKSIGLSKDDAAPRIFSLAHQTVHKYGKNSFAWEGTTPKVIITDPEQIKEVFNKIQDFPKPKLNPIAKYISIGLVHYEGDKWAKHRKIINPAFHLEKLKGMLPAFSHSCHEMISKWKGLLSVDGTCEVDVWPFLQNLTCDVISRTAFGSSYAEGAIIFELLKRQGYALMTARYARIPLWWLLPSTTKRRMKEIERGIRDSLEGIIRKREKALKSGKSTDDDLLGILLQSNHIENKGDENSKSAGMTTQEVMEECKLFYLAGQETTAALLAWTMVLLGKHPEWQARARQEVLQVFGNQNPNFEGLGRLKIVTMILYEVLRLYPPGIYLTRALQKDLKLGNLLLPAGVQVSVPILLIHHDEGIWGNDAKEFNPERFAEGIAKATKGQVCYFPFGWGPRICVGQNFALLEAKIVLSLLLQNFSFELSPSYAHVPTTVLTLQPKHGAPIILHKL

SEQ ID NO: 12 - DNA, 1572 bp, Glycyrrhiza glabra
  source 1..1572: mol_type = other DNA
atggatgcatcttccacaccaggggctatctgggttgttctaacagtgatactagctgcgattcccatatgggcatgccatatggtcaacacgctgtggctgaggccaaagaggttggaaaggcatctcagagctcaaggtcttcatggtgacccttacaagctctcacttgacaactccaagcaaatctatatgctcaagttgcaacaagaagcacaatcaaaatccattggtctctccaaagatgatgctgcaccacgaatcttctcccttgcccatcaaactgtacacaaatatggaaagaactcctttgcatgggaagggacaacaccaaaggtgatcatcacagacccagagcaaattaaggaagtctttaacaagattcaggacttccccaaaccaaaattaaatcccatcgccaagtatattagcatcggtctagtacattatgagggtgacaaatgggccaaacatcgaaagattatcaatccggcattccacttagaaaaattgaaaggtatgctgccagcattttctcatagctgccatgaaatgattagcaaatggaaggggttattgtcagtagatggaacgtgtgaggttgatgtttggcccttccttcaaaatctcacttgtgatgtaatttctaggacggcattcggaagcagctatgcagaaggagcaataatatttgaacttttgaaaaggcagggatatgctttgatgacagcacgatacgcgcgcattccattatggtggcttctaccatcaactaccaaaaggaggatgaaggaaattgaaagaggcatacgtgattcacttgaaggtatcattagaaaacgagaaaaagcattgaagagtggcaaaagcaccgatgacgacttattaggcatacttttgcaatcaaatcacattgaaaataaaggagatgaaaacagtaagagtgctggaatgaccacccaagaagtaatggaggaatgcaaacttttttacctggcagggcaagagaccaccgcggctttgctggcctggacaatggtgttattaggcaagcatcctgaatggcaagcacgtgcaaggcaggaagttttgcaagtttttgggaatcaaaatccaaacttcgaagggttaggtcgcctcaaaattgtaaccatgattttatatgaggtactcaggctgtacccacctgggatttacctcacccgagctcttcaaaaggatttgaaacttggaaaccttttgctacctgctggagtacaggtttccgtaccaatacttttgattcaccatgatgaaggtatatggggcaatgatgcaaaggagttcaatcctgaaaggtttgctgaaggaattgcaaaggcaacaaaaggccaagtttgctatttcccttttggatggggtcctagaatatgtgttgggcaaaactttgccttattagaagccaagattgtattgtcattgctgctgcagaatttctcatttgagttatctccgagttatgcacatgttcctaccacggtgcttactttgcagccaaaacatggggcacccatcattctgcataaactgtaa

SEQ ID NO: 13 - protein, 524 aa, Medicago truncatula
MEVFMFPTGTTVIISVLSVLLAVIPWYLLNKLWLKPKRFEKLLKAQGFQGEPYNLSVLKDKSKQNYMLKLQQEDKSKSIGLSKEAAPSIFTPVHQTVRKYGNNSFLWEGTTPRVIITDPDQIKDVFNKIDDFPKPKLRSIAKYLSVGILDHEGKKWAKHRKIANPAFHLEKLKVMLPAFSHSCNEMISKWKELLSSDGTCEIDVWPSLQNFTCDVISRTAFGSSYAEGTKLFQLLKKQGFLLMTGRHTNNPLWGLLATTTKTKMKEIDREIHDSLEGIIEKREKALKNGETTNDDLLGILLQSNHAEKQGQGNSKNIGMTTQDVIDECKLFYLAGQETTSSLLVWTMVLLGRYPEWQARAREEVLQVFGNQNPNNEGLSQLKIVTMILYEVLRLFPPLIYFNRALRKDLKLGNLLLPEGTQISLPILLIHQDHDLWGDDAKEFKPERFAEGIAKATKGQVSYFPFGWGPRICLGQNFALLEAKIAVSLLLQNFSFELSPNYVHVPTTVLTLQPKNGASIILHKL

SEQ ID NO: 14 - DNA, 1575 bp, Medicago truncatula
  source 1..1575: mol_type = other DNA
atggaagtgtttatgtttcccacaggaacaacagtaatcatctctgttctttcagttctacttgctgtgattccatggtatcttctcaacaagttatggcttaagccaaagaggtttgagaaacttctcaaagctcaaggttttcaaggtgaaccttataacctttcagtattaaaggacaaatcaaaacaaaattatatgttgaagttgcaacaagaagataaatctaaatccattggtctctccaaagaagctgcaccgtctatcttcactcctgttcatcaaactgtacgcaaatatggaaacaattcctttttatgggaaggtacaacaccaagggttatcatcacagaccctgatcaaattaaggatgtatttaacaagattgatgacttccccaaaccaaaactaagatccatcgccaagtatttgagcgttggtatactagatcatgagggtaagaaatgggctaaacataggaagatcgccaatccagcattccacctagaaaaattgaaagttatgctgcctgcattttctcacagttgcaatgaaatgataagcaaatggaaggaactattgtcatcagatggaacatgtgagattgatgtttggccttcccttcagaattttacctgtgatgtaatttctcggacggcatttggaagcagctacgcagaaggaacaaaactatttcaacttctaaagaagcagggatttcttttgatgacagggcgacacacgaacaatccattatgggggcttctagcaacaactaccaagacgaagatgaaagaaattgatagagaaatccatgattcacttgagggaatcattgaaaaacgagaaaaagcactgaagaatggtgaaaccaccaatgacgatttattaggcattcttttgcaatcaaatcatgccgaaaaacaaggacaaggaaatagtaagaatattgggatgaccacccaagatgtgatagatgaatgcaaattgttttaccttgctgggcaagagacgacttcaagtttgctggtttggacaatggtgttattaggcaggtatcctgaatggcaagcacgtgcaagggaggaagttttgcaagtttttgggaaccaaaatcctaacaacgaaggattaagtcaacttaaaattgttaccatgattttgtacgaggtactaaggttattcccacctttaatttacttcaaccgagctcttcgaaaggatttgaaacttggaaaccttttgctacctgaaggaacacaaatttccctaccaatactattgattcaccaagatcatgatctatggggtgatgatgcaaaggagttcaaacctgaaaggtttgctgaaggaattgcgaaggcaacaaaaggacaagtttcttatttcccttttggatggggtcctagaatttgtcttggacaaaactttgccttattagaagcaaagatagcagtatcattgttgctgcagaatttctcattcgaactttctccaaattatgtgcatgttcctaccacggtgcttactttgcagccaaaaaatggggcaagcatcattttgcataaactgtaa

SEQ ID NO: 15 - protein, 506 aa, Glycyrrhiza uralensis
MDSFGVEGDHQADTTVLKAVFLPFISKSHLIRVVDKARIFAMHGVDVTIITTPANAAAFQTSIDHDSSRSRSIKTHIVPFPQVPGLPQGFERLDADTPQHLLPKIYQGLSILQEQFQQLFREMRPDFIVTDMYYPWSVDAAAELGIPRLVCNGGSYFAQSAVNSIELFSPQAKVDSNTETFLLPGLPHEVEMTRLQLPDWLRGAPNEYTYLMKMIKDSERKSYGSLFNSFYELEGTYEEHYKKAMGTKSWSVGPVSLWVNQDASDKACRGDVKEGKGDGVVLTWLDSKTEDSVLYVSFGSMNKFPKTQLVEIAHALEDSGHDFIWVVGKIEEGEGGADFLREFEKKVKEKNRGYLIWGWAPQLLILEHPAVGAVVTHCGWNTVMESVNASLPLATWPLFAEQFFNEKLVVDVVKIGVPVGVKEWRNWNEFGDEVVKREDIGKAIAFLMGGGDESLEMRKRVKVLSGATKKAIQVDGSSYTKLKELIEELKSIKLQKVNNKLMEAVA

SEQ ID NO: 16 - DNA, 1521 bp, Glycyrrhiza uralensis
  source 1..1521: mol_type = other DNA
atggactcctttggggttgaaggtgatcaccaagccgacaccacagtgctgaaggcggtttttcttcccttcatctcgaaaagtcatctcatccgtgtggtggacaaagcaaggatcttcgccatgcacggcgtggatgtcaccatcatcactacgccggccaacgctgccgctttccaaacctccattgaccacgactccagccgcagccgttccatcaaaacgcacatcgttccgttcccccaagtacccggtctgccacagggattcgagagactcgatgccgacactcctcaacacttgctccccaagatctaccaggggctatccattctgcaagagcaattccaacaactgttccgtgaaatgagaccagatttcatagtcactgacatgtactacccttggagcgtcgatgccgccgccgagttggggattccgaggttggtttgtaacggtggaagctacttcgctcagtcagctgttaactccattgagctattttcaccacaagccaaggttgattcaaataccgagacttttctgcttcctgggttaccccatgaggttgagatgacacgtttgcaactaccggattggcttagaggagcaccgaatgagtacacctatttgatgaagatgatcaaggattcagagaggaagagttatgggtcattgttcaatagcttttatgagcttgaagggacttatgaggagcattacaagaaagccatgggaaccaagagttggagtgtggggccagtttctttgtgggtgaaccaagatgcttctgataaggcttgtaggggggatgttaaagaaggaaaaggagatggggtggtgcttacttggctggattctaaaacagaggactctgttttgtatgtgagttttgggagcatgaacaagttccctaaaactcagcttgttgagatagctcatgccctcgaagattctggccatgatttcatttgggtcgttggcaaaattgaagaaggtgaaggtggtgctgattttttgagggaatttgagaagaaagtgaaagaaaaaaacagaggttatctgatatggggttgggcaccacagcttctgattctggagcatcctgcggttggagcagtggtgactcattgtgggtggaacaccgttatggaaagtgtgaatgcaagtttgccattggcaacttggccattgtttgcggagcagttcttcaatgagaagctagtggttgatgtggtgaagattggtgtgccagttggggttaaggaatggagaaattggaatgagtttggggatgaggttgtgaagagggaggacataggaaaggccattgcttttttgatgggtggtggagatgagtccttggaaatgaggaagagggtcaaggtgctcagtggtgctacaaagaaggctattcaggttgatgggtcttcttacaccaagttgaaagaactcattgaggagctcaagtcaattaagcttcaaaaggtcaacaacaaattaatggaggcagtggcttaa

SEQ ID NO: 17 - DNA, 37 bp, synthetic construct
  misc_feature 1..37: note = primer
  source 1..37: mol_type = other DNA
aaaaagcaggctatggcaatgttcacgtaccacgttg

SEQ ID NO: 18 - DNA, 45 bp, synthetic construct
  misc_feature 1..45: note = primer
  source 1..45: mol_type = other DNA
agaaagctgggtctatccacttttgcttttcattgttactatggc

SEQ ID NO: 19 - DNA, 25 bp, synthetic construct
  misc_feature 1..25: note = primer
  source 1..25: mol_type = other DNA
caccatggcaagcttcacccttcac

SEQ ID NO: 20 - DNA, 23 bp, synthetic construct
  misc_feature 1..23: note = primer
  source 1..23: mol_type = other DNA
ttatccactcttgcttttcatgg

SEQ ID NO: 21 - DNA, 37 bp, synthetic construct
  misc_feature 1..37: note = primer
  source 1..37: mol_type = other DNA
gggcggccgcactagaaaaatggtgaagatatgttgt

SEQ ID NO: 22 - DNA, 33 bp, synthetic construct
  misc_feature 1..33: note = primer
  source 1..33: mol_type = other DNA
atccatcgatactagttaggcaacggcaggcat

SEQ ID NO: 23 - DNA, 15 bp, synthetic construct
  misc_feature 1..15: note = synthetic
  source 1..15: mol_type = other DNA
gggcggccgcactag

SEQ ID NO: 24 - DNA, 15 bp, synthetic construct
  misc_feature 1..15: note = synthetic
  source 1..15: mol_type = other DNA
atccatcgatactag

SEQ ID NO: 25 - DNA, 1443 bp, Arabidopsis thaliana
  source 1..1443: mol_type = other DNA
atggtgaagatatgttgtattggagctgggtatgttggtggaccaacaatggcagtgattgcattgaaatgtccagacgttgaagtagcggttgttgatatctctgtaccacgtatcaacgcttggaacagtgacacgcttccgatttacgagcctggtcttgatgatgttgtgaagcaatgccgtggcaagaaccttttctttagtactgatgttgagaaacatgttagggaagctgatattgtgtttgtttctgtcaacacaccgactaagactagaggtcttggtgctggtaaagctgcggatcttacgtactgggagagcgctgcgcgtatgatcgctgatgtttcggtatcggataagattgtcgttgagaaatcgactgttccggttaaaacagctgaagctattgagaagattttgacacataacagtaaagggattaagtttcagattctttcgaatcccgagtttttggcggaaggaaccgcgattaaggacctatttaatccggaccgtgttcttatcggagggcgggaaaccccagaagggtttaaagcggtgcagactctcaagaatgtgtatgcacactgggttcctgaaggccaaatcataacaaccaatctctggtctgctgagctgtccaagcttgcggcaaacgctttcttggctcaaaggatttcatcagtgaatgctatgtcggctctgtgtgaagccacaggcgcagatgtcacgcaagtgtcttacgcggttggtacagactcaaggattggtcccaagttcttgaactcgagtgttggattcggtggttcgtgtttccagaaggacattctgaatcttgtctacatctgtgagtgcaacggactcccggaagtggcagagtactggaagcaagtcatcaagatcaatgactaccagaagagccggttcgtgaaccgtgttgtttcctccatgttcaactctgtatcaaacaagaagattgcggttctcggtttcgcattcaagaaagacaccggtgacacaagggagactccagccatcgatgtgtgcaagggtcttttagaagacaaagcaaggctaagcatttacgacccacaagtgactgaggatcagatccagagggatttatccatgaacaagttcgactgggaccatcctctacatttgcagccaatgagcccaacaacagtgaaacaagtgaccgttacttgggacgcatacgaagcaactaaggacgctcacggtatctgcatcatgaccgagtgggatgagttcaagaaccttgatttccagaagatctttgacaacatgcagaaaccagctttcgtgttcgatggaagaaacattatgaatctgcaaaagctaagggagattggtttcattgtttactccattggtaagcctctcgacgactggctcaaggacatgcctgccgttgcctaa

SEQ ID NO: 26 - DNA, 24 bp, synthetic construct
  misc_feature 1..24: note = primer
  source 1..24: mol_type = other DNA
gcggctaggcttgcgctcaatttt

SEQ ID NO: 27 - DNA, 24 bp, synthetic construct
  misc_feature 1..24: note = primer
  source 1..24: mol_type = other DNA
tgcctgtttggacacacaaccgtg

SEQ ID NO: 28 - DNA, 24 bp, synthetic construct
  misc_feature 1..24: note = primer
  source 1..24: mol_type = other DNA
cgttggttgctgctggcctaacac

SEQ ID NO: 29 - DNA, 25 bp, synthetic construct
  misc_feature 1..25: note = primer
  source 1..25: mol_type = other DNA
ccgccaaagaagcaaacaatgttca

SEQ ID NO: 30 - DNA, 25 bp, synthetic construct
  misc_feature 1..25: note = primer
  source 1..25: mol_type = other DNA
ccatggcggttccgtgaatcttagg

SEQ ID NO: 31 - DNA, 33 bp, synthetic construct
  misc_feature 1..33: note = primer
  source 1..33: mol_type = other DNA
aaaaagcaggctatggcaagcttcacccttcac

SEQ ID NO: 32 - DNA, 35 bp, synthetic construct
  misc_feature 1..35: note = primer
  source 1..35: mol_type = other DNA
agaaagctgggtttatccactcttgcttttcatgg

SEQ ID NO: 33 - DNA, 33 bp, synthetic construct
  misc_feature 1..33: note = primer
  source 1..33: mol_type = other DNA
aaaaagcaggctatggccaatttcactctccac

SEQ ID NO: 34 - DNA, 35 bp, synthetic construct
  misc_feature 1..35: note = primer
  source 1..35: mol_type = other DNA
agaaagctgggtctacccactcttcattttcatag

SEQ ID NO: 35 - DNA, 12 bp, synthetic construct
  misc_feature 1..12: note = synthetic
  source 1..12: mol_type = other DNA
aaaaagcaggct

SEQ ID NO: 36 - DNA, 12 bp, synthetic construct
  misc_feature 1..12: note = synthetic
  source 1..12: mol_type = other DNA
agaaagctgggt

SEQ ID NO: 37 - DNA, 22 bp, synthetic construct
  misc_feature 1..22: note = primer
  source 1..22: mol_type = other DNA
caccatggccaatttcactctc

SEQ ID NO: 38 - DNA, 23 bp, synthetic construct
  misc_feature 1..23: note = primer
  source 1..23: mol_type = other DNA
ctacccactcttcattttcatag

SEQ ID NO: 39 - DNA, 25 bp, synthetic construct
  misc_feature 1..25: note = primer
  source 1..25: mol_type = other DNA
caccatggcagaattcagtcttcac

SEQ ID NO: 40 - DNA, 23 bp, synthetic construct
  misc_feature 1..23: note = primer
  source 1..23: mol_type = other DNA
ttacccactcttgcttttcatag

SEQ ID NO: 41 - DNA, 2097 bp, Astragalus sinicus
  source 1..2097: mol_type = other DNA
atggcagaattcagtcttcacacagaaactgttcaatcatggttaccactaagcagagctcacataatattccacttcgcatgcgtcttattactcttctactacagaatcacacgtttcataccaacactaccatccattcttatgatagcagctgagtttatcctttctgtcttatggttcttcaaccaagcatacaggtggcgtcctgttagcaggtctgtcaaaacacaaaacttaccaaaagatgaaaatcttccaggattggatatatttgtgtgcaccattgatcctgagaaggaacctacggttgaagttatgaatactcttgtttctgctatttcaatggattatcctcctcataaactttctgtttatctttctgatgatggagctgctcctgttactttttatgggattaaagaggcttctgaatttgcaaaggtttgggtacctttttgtaataaatatggtgttaagtctaggtgtcctaaggttttcttctctcctcttgctgaagatgaacttgttcctctaaggacacatcaattcgaggatgacaaagaggacatcaaggttaaatacgacaaaatgcagaaaaacattgaaaaatttggttcagaaccaaaaaatgtcagtatggtgaccgacagacctgctcggatcgagattataagtgatgaaccagaaatggcacgtgttgtttatgtgtctcgtgaaagaagaccatcacttgctcacaagttcaaaggaggtgctctgaatacattgctaagagtgtcaggtctaatcagcaatggaggttatgtacttgcagtggattgtgatatgtactgcaatgatccaacctcagccaaacaagccatgtgtttctttcttgatcctgaatcctctaaatatattgcatttgtccaattccctcaaatgtttcacaaccttagcaaaaaagacatctatgataatcaatctagaactgcttttaagacaatgtggcaagggatggatggtttgagaggtccaggtctttctggcagtggcaattacttgagtagagctgctttgttatttggaagtccaaatcaaaaagatgactatcttctagatgctgaaaactactatggcaagtctagcacatacatagaatcactgagggaaatccgtggacaagaagaacaaactaacaaaaagaataatctttcaagagaacaaattttaagagaagctcaagtagtagcctcttgttcctatgagacaaacacaaaatggggaacagaggtaggattctcatatgggatattactagagagtaccataactgggtatctattgcattctagaggatggaaatcagcatatctatacccaaaaacaccatgtttcttaggctgtgctcctactgacatcaaggaagggatgcttcaattggtcaagtggttgtctgaactttgcttgcttgcagtctctaaatacagcccttttacctatggattttcaacaatctccactattcataacttcacttattgcttcatgtctatttcttccttatatgctattggcttcatcctttatggcattgtacctcaagtttgcttcctcaaaggaatacctgtctttccaaaggtcacagatccttggtttctagtgtttgcaatactatatatagcgacacagattcaacatttgatagaggttatttctggggatggatcagtgtcaatgtggtgggatgaacaaagaatttggattctcaagtcagttactagcctatttgcgatgatagaaggagttaagaaatggttgggattgaacaagaaaaaattcaatttgtcaaataaagctgttgacacagacaaagagaaaataaaaaaatatgaacaagggaggtttgattttcaaggtgcagctttgtatatgtctccaatggttgtgctgctgatagtcaacacagtttgcttctttggtggtttgtggagactattgagtactagagattttgaagatatgtttggtcaactattcctacttggttatgtaatggctcttagttatccaatatttgagggcataataactatgaaaagcaagagtgggtaa

SEQ ID NO: 42 - protein, 698 aa, Astragalus sinicus
MAEFSLHTETVQSWLPLSRAHIIFHFACVLLLFYYRITRFIPTLPSILMIAAEFILSVLWFFNQAYRWRPVSRSVKTQNLPKDENLPGLDIFVCTIDPEKEPTVEVMNTLVSAISMDYPPHKLSVYLSDDGAAPVTFYGIKEASEFAKVWVPFCNKYGVKSRCPKVFFSPLAEDELVPLRTHQFEDDKEDIKVKYDKMQKNIEKFGSEPKNVSMVTDRPARIEIISDEPEMARVVYVSRERRPSLAHKFKGGALNTLLRVSGLISNGGYVLAVDCDMYCNDPTSAKQAMCFFLDPESSKYIAFVQFPQMFHNLSKKDIYDNQSRTAFKTMWQGMDGLRGPGLSGSGNYLSRAALLFGSPNQKDDYLLDAENYYGKSSTYIESLREIRGQEEQTNKKNNLSREQILREAQVVASCSYETNTKWGTEVGFSYGILLESTITGYLLHSRGWKSAYLYPKTPCFLGCAPTDIKEGMLQLVKWLSELCLLAVSKYSPFTYGFSTISTIHNFTYCFMSISSLYAIGFILYGIVPQVCFLKGIPVFPKVTDPWFLVFAILYIATQIQHLIEVISGDGSVSMWWDEQRIWILKSVTSLFAMIEGVKKWLGLNKKKFNLSNKAVDTDKEKIKKYEQGRFDFQGAALYMSPMVVLLIVNTVCFFGGLWRLLSTRDFEDMFGQLFLLGYVMALSYPIFEGIITMKSKSG

SEQ ID NO: 43 - DNA, 37 bp, synthetic construct
  misc_feature 1..37: note = primer
  source 1..37: mol_type = other DNA
aaaaagcaggctatggcaacgttcacataccacgtag

SEQ ID NO: 44 - DNA, 35 bp, synthetic construct
  misc_feature 1..35: note = primer
  source 1..35: mol_type = other DNA
agaaagctgggtctatccactcttgcttttcattg

SEQ ID NO: 45 - DNA, 34 bp, synthetic construct
  misc_feature 1..34: note = primer
  source 1..34: mol_type = other DNA
aaaaagcaggctatggcgaccttccacacagaaa

SEQ ID NO: 46 - DNA, 35 bp, synthetic construct
  misc_feature 1..35: note = primer
  source 1..35: mol_type = other DNA
agaaagctgggtctattgcaccttgcttttcatgg

SEQ ID NO: 47 - DNA, 2100 bp, Glycine max
  source 1..2100: mol_type = other DNA
atggcaacgttcacataccacgtagaaacagttcaatcattcttagccctaagcagacttcacatactcatccacttagtggcagtgttgtctctctgttactaccgcatcacccacttcttcctccaacctccaacagcgccatggcttctcatgactgctgcggagcttcttctctcgctgctctggttcttcaaccaagccttccggtggcgaccggtgtcgcggagcgtcatgactgagaagcttccgagtgaggagaagctgccggggcttgacatcttcgtgtgcacgctcgatcccgagaaggagcccaccgtggaggtcattgacaccatcatctccgccgtgtccatggattacccctccgacaagctttccgtctatctctccgacgacggcgggtgcgacgtgactctctatgggatccgagaggctgctgagttcgccaaggagtgggttccgttctgtaaaaagtatggggtgaagtcaaggtgtcccaaagtcttcttctctccctttggggatgaggatcaagagactcttcgggacgatcaattcagaacacagagagacctcgtcaaggctaaatacgagaaaatgcagaaaaatatcgagaaatttggttcagaccctaaaagtcgtcgtactgtgagtgacagacaacctcgtattgagattataaatgaccaaccgggaatgccacttattgtttatgtgtctcgcgaaagaaggccatcccttcctcacaaattcaaaggaggagccgtcaacacattgctcagagtctcaggtttaatcaataatggaccttatgttctagtaatggactgtgatatgtatagcaatgatccaacctctgccaaacaagccatgtgcttctttcttgatcctgaaacgtccaaatatattgcatttgttcaattccctcaaatgtttcacaaccttagcaaaaaggacatttatgatagtcaagctagaactgcttttaagacaatgtggcaaggcatggatggactaagaggtccaggtctttctggcagcggcaattacttaagtagaagtgctttactatttggaagtccaaaccaaaaagatgactatctgcaagatgcacaaaagtacttcggcaagtctaccgcctacattgaatcactgaaggccatccgtggacagaaaagtagcaaaaagaatatttcaagagatgaaatgttaagagaagctcaagtagtggcctcttgttcctacgaaaataacacaaattggggcacagaggtgggattctcatatggcatattactagagagttctattactggctatattcttcacagcagaggatggaaatcagcatatctttacccgaaaacaccatgtttcttaggttgtgctcccactgacatcaaggaaggcatgcttcagttggttaagtggttgtctgaacttttgttgcttggtgtctcctccaaatacagcccattcacttatggattttcaagaatgtccatacttcacacattcacttattgcttcatcacaatgtcatccctttatgctgttgtcttcatcctctacggcattgtacctcaagtatgcctcctcaaaggaatccctgtgtttccaaaggccacagacccttggtttgccgtgtttgcatttgtgtatgtatctactcagattcaacatttgattgaggttctctctggggatggatctgtgacaatgtggtgggatgaacaaagaatttggattctgaagtcagttaccagcatatttgcaattatagatggagtcaagaagtggttggggttgagcaaggtgaaattcaacttgtcaaacaaagcaattgacaaagagaagctcaagaaatatgagcaaggtagattcgatttccaaggtgcagctgtgttcatggctccattggttctattactcacagccaacattgttagcttcttagttggtatatggagactatttaatttcaatgtgaaggattttgaagaaatgtttggtcaacttttcctagttacctatgtaatggttcttagttatccacttcttgaggccatggtaacaatgaaaagcaagagtggatag

SEQ ID NO: 48 - protein, 699 aa, Glycine max
MATFTYHVETVQSFLALSRLHILIHLVAVLSLCYYRITHFFLQPPTAPWLLMTAAELLLSLLWFFNQAFRWRPVSRSVMTEKLPSEEKLPGLDIFVCTLDPEKEPTVEVIDTIISAVSMDYPSDKLSVYLSDDGGCDVTLYGIREAAEFAKEWVPFCKKYGVKSRCPKVFFSPFGDEDQETLRDDQFRTQRDLVKAKYEKMQKNIEKFGSDPKSRRTVSDRQPRIEIINDQPGMPLIVYVSRERRPSLPHKFKGGAVNTLLRVSGLINNGPYVLVMDCDMYSNDPTSAKQAMCFFLDPETSKYIAFVQFPQMFHNLSKKDIYDSQARTAFKTMWQGMDGLRGPGLSGSGNYLSRSALLFGSPNQKDDYLQDAQKYFGKSTAYIESLKAIRGQKSSKKNISRDEMLREAQVVASCSYENNTNWGTEVGFSYGILLESSITGYILHSRGWKSAYLYPKTPCFLGCAPTDIKEGMLQLVKWLSELLLLGVSSKYSPFTYGFSRMSILHTFTYCFITMSSLYAVVFILYGIVPQVCLLKGIPVFPKATDPWFAVFAFVYVSTQIQHLIEVLSGDGSVTMWWDEQRIWILKSVTSIFAIIDGVKKWLGLSKVKFNLSNKAIDKEKLKKYEQGRFDFQGAAVFMAPLVLLLTANIVSFLVGIWRLFNFNVKDFEEMFGQLFLVTYVMVLSYPLLEAMVTMKSKSG

SEQ ID NO: 49 - DNA, 2091 bp, Glycine max
  source 1..2091: mol_type = other DNA
atggcgaccttccacacagaaaccgtgcaatcagggttggccttgagcagactccacatcctattccactcggtggcactcttgtttctctattactaccgcataagccacatcttactggaaccaagctttgtatggattttcatgaccatagcggagcttatcttcggcgagctctggctcttcaaacaggcgttccggtggcggcccgtgtcgagggccgtcatgccggagaagctgccgagcgacggcaagcttccggcgctcgacatcttcgtctgcacggttgaccccgaaaaggagccgacggtgcaggtgatggacaccgtcatctccgccattgccatggactacccctccaacaagctcgccgtgtacctttccgacgatggcgggtgtccggtgactctgtatgggatcagagaggcttctcggttcgcaaaggagtgggttccgttctgcagaaagtatgggatcaattcacggtgccccaaggccttcttctctcccatgggggaggatgaacgtgaactgcttcttcttcgcaaccatgaattcttggcagagcaagaacaactcaaggctaaatacaatataatgcaaaaaaatattgacgaatttggaagagaccctaaaaatcgttccattgtgtttgatagaccagctcgcattgagattataaatgagcaatccgaaataccactggttgtttatgtgtctcgtgaaagaaggccaaatgttcctcatacatacaaagggggagccctcaacacattgctcagagtctcagggctattcagtaacgggccctatgtacttgtagttgattgtgatatgtattgcaatgatccatcatcagctaaacaagccatgtgcttttttcttgatcctgaaacctccaaagatattgcttttgtccaattccctcaaatgtttcacaaccttagcatgaaagacatctacgatagtcaacataggcatgcttttacaacaatgtggcaaggaatggatggactaagaggtccaggtctttctggtagtggcaattacttaagtagaagtgcattaatctttccaagcccatatgaaaaagacggctatgaacataatgcccaaaacaaatttggcaactctaccatgtacattgaatcattaaaggccattcaaggacaacaaacttataaaacgagcatttcaagaaatgtgattttacaggaagcacaagcagtggcctcttgttcctatgaaatagacacaaattggggtaatgaggtaggattctcatatgttatattactggagagtacagttactggctatcttcttcactgtagaggatggagatcaacttacctttaccccaaaagaccttgtttcttgggatgtgcccccactgacttcatggaaggcatgcttcagttggtgaaatggagttctgaacttttcttgctaggaatatccaaatacagccctttcacttatgggatttcaagaattcctattctgcacaactttaccttttgctacttcacatctacatgtcaatatattgttgccttaatagtatatggcatcattcctcaagtatgcttcttgaaaggaactcctgtgtttcctaaggttacagaaccatggtttgtagtttttgcaatattatatgtatcctctcaaagtcaacatttgattgaagtcctttatggtggtggctctttgggaacatggtgggatgaacaaagaatatggattgtaaagtcaattgttggaggcatatttggatctatactagcaatcaagaaacgttttgggttaaacaaagcaaaattcattttatcaaataaagttgttgccaaagagaagtttgagaaatatgaacaaggtaagttcgagttcgaaggtgcagctttgttcatgtctccattggttggattactcatagtgaatattctttgcttctttggtggtttatggagactatttaatgtgaaagattttgaaaagatgtctggccaactttttctacttggctatctggcggcgctcagttatcccatttttgaggggataataaccatgaaaagcaaggtgcaatag

SEQ ID NO: 50 - protein, 696 aa, Glycine max
MATFHTETVQSGLALSRLHILFHSVALLFLYYYRISHILLEPSFVWIFMTIAELIFGELWLFKQAFRWRPVSRAVMPEKLPSDGKLPALDIFVCTVDPEKEPTVQVMDTVISAIAMDYPSNKLAVYLSDDGGCPVTLYGIREASRFAKEWVPFCRKYGINSRCPKAFFSPMGEDERELLLLRNHEFLAEQEQLKAKYNIMQKNIDEFGRDPKNRSIVFDRPARIEIINEQSEIPLVVYVSRERRPNVPHTYKGGALNTLLRVSGLFSNGPYVLVVDCDMYCNDPSSAKQAMCFFLDPETSKDIAFVQFPQMFHNLSMKDIYDSQHRHAFTTMWQGMDGLRGPGLSGSGNYLSRSALIFPSPYEKDGYEHNAQNKFGNSTMYIESLKAIQGQQTYKTSISRNVILQEAQAVASCSYEIDTNWGNEVGFSYVILLESTVTGYLLHCRGWRSTYLYPKRPCFLGCAPTDFMEGMLQLVKWSSELFLLGISKYSPFTYGISRIPILHNFTFCYFTSTCQYIVALIVYGIIPQVCFLKGTPVFPKVTEPWFVVFAILYVSSQSQHLIEVLYGGGSLGTWWDEQRIWIVKSIVGGIFGSILAIKKRFGLNKAKFILSNKVVAKEKFEKYEQGKFEFEGAALFMSPLVGLLIVNILCFFGGLWRLFNVKDFEKMSGQLFLLGYLAALSYPIFEGIITMKSKVQ

SEQ ID NO: 51 - DNA, 12 bp, synthetic construct
  misc_feature 1..12: note = synthetic; 12 nucleotides added to the 5' terminal of the forward primer
  source 1..12: mol_type = other DNA
aaaaagcaggct

SEQ ID NO: 52 - DNA, 12 bp, synthetic construct
  misc_feature 1..12: note = synthetic; 12 nucleotides added to the 5' terminal of the reverse primer
  source 1..12: mol_type = other DNA
agaaagctgggt